SEQUENCE LISTING (14 sequences)

SEQ ID NO: 1 - protein, 725 aa, Homo sapiens [sequence omitted]

SEQ ID NO: 2 - protein, 794 aa, Mus musculus
MSFPKAPLKRFNDPSGCAPSPGAYDVKTSEATKGPVSFQKSQRFKNQRESQQNLSIDKDTTLLASAKKAKKSVSKKDSQKNDKDVKRLEKEIRALLQERGTQDKRIQDMESELEKTEAKLNAAVREKTSLSASNASLEKRLTELTRANELLKAKFSEDGHQKNMRALSLELMKLRNKRETKMRSMMVKQEGMELKLQATQKDLTESKGKIVQLEGKLVSIEKEKIDEKCETEKLLEYIQEISCASDQVEKCKVDIAQLEEDLKEKDREILSLKQSLEENITFSKQIEDLTVKCQLLETERDNLVSKDRERAETLSAEMQILTERLALERQEYEKLQQKELQSQSLLQQEKELSARLQQQLCSFQEEMTSEKNVFKEELKLALAELDAVQQKEEQSERLVKQLEEERKSTAEQLTRLDNLLREKEVELEKHIAAHAQAILIAQEKYNDTAQSLRDVTAQLESVQEKYNDTAQSLRDVTAQLESEQEKYNDTAQSLRDVTAQLESEQEKYNDTAQSLRDVTAQLESVQEKYNDTAQSLRDVSAQLESYKSSTLKEIEDLKLENLTLQEKVAMAEKSVEDVQQQILTAESTNQEYARMVQDLQNRSTLKEEEIKEITSSFLEKITDLKNQLRQQDEDFRKQLEEKGKRTAEKENVMTELTMEINKWRLLYEELYEKTKPFQQQLDAFEAEKQALLNEHGATQEQLNKIRDSYAQLLGHQNLKQKIKHVVKLKDENSQLKSEVSKLRSQLVKRKQNELRLQGELDKALGIRHFDPSKAFCHASKENFTPLKEGNPNCC

SEQ ID NO: 3 - protein, 713 aa, Rattus norvegicus
MSFPKAPLKRFNDPSGCAPSPGAYDVKTSESTKGPVSFQKSQRFKNQRESQQNLNIDKDTTLLASAKKAKTLVSKKESQKNDKDVKRLEKEIHVLLQERGTQDKRIQDMESELENTEANLNAPVTEKPSLSANNASLEKRLTELTRANELLKSKFSEDAHQKNMRTLSLELMKLRNKRETKMRSMMAKQEGMELKLQATQKDLIESKGKIVQLEGKLVSIEKEKIDEKSETEKLLEYIEEISCASDQVEKYKLDIAQLEEDLKEKDREILCLKQSLEEKVSFSKQIEDLTVKCQLLEAERDDLVSKDRERAESLSAEMQVLTEKLLLERQEYEKLQQNELQSQSLLQQEKELSAHLQQQLCSFQEEMTSERNVFKEQLKLALDELDAVQQKKEQSEKLVKQLEEETKSTAEQLRRLDDLLREKEIELEKRTAAHAQATVIAQEKYSDTAQTLRDVTAQLESYKSSTLKEIEDLKLENLTLQEKVAMAEKRVEDVQQQILTAESTNQEYAKVVQDLQNSSTLKEAEIKEITSSYLEKITDLQNQLRQQNEDFRKQLEEEGAKMTEKETAVTELTMEINKWRLLYEELFDKTKPFQQQLDAFEAEKQALLNEHGATQEQLSKIRDSYAQLLGHQNLKQKIKHVVKLKDENSQLKSEVSKLRSQLAKRKQNELRLQGELDKALGIRHFDPSKAFCHESKENVTLKTPLKEGNPNCC

SEQ ID NO: 4 - DNA, 3114 bp, Homo sapiens
ccgccagtgtgatggatatctgcagaattcggcttactcactatagggctcgagcggccgcccgggcaggtgtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagaaggaatctcaaaagaatgataaagatttgaagatattagagaaagagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggaaaatattgttatattatctaaacaagtagaagatctaaatgtgaaatgtcagctgcttgaaaaagaaaaagaagaccatgtcaacaggaatagagaacacaacgaaaatctaaatgcagagatgcaaaacttaaaacagaagtttattcttgaacaacaggaacatgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggcttttctagtctgagcttctttagctaggctaaaacaccttggcttgttattgcctctactttgattcttgataatgctcacttggtcctacctattatcctttctacttgtccagttcaaataagaaataaggacaagcctaacttcatagtaacctctctattttaatcagttgtttaataatttacaggttcttaggctccatcctgtttgtatgaaattataatctgtggattggcctttaagcctgcattcttaacaaactcttcagttaattcttagatacactaaaaatctgaagaaactctacatgtaactatttcttcagagtttgtcatatactgcttgtcatctgcatgtctactcagcatttgattaacatttgtgtaataagaaataaaattacacagtaagtcatttaaccaaaaaaaaaaaaaaaaaaaaaaaaaaaaaaaaaaaaaaaaaaaaaaaaaaa

SEQ ID NO: 5 - DNA, 2479 bp, Mus musculus
ctgtctcatctggacccaggcgtcagaatgtcctttcctaaggcgcccctgaagagattcaatgacccttcgggttgtgctccatctccgggtgcttatgatgttaaaacttcagaagcaactaaaggaccagtgtcttttcagaaatcacaaagatttaaaaaccaaagagagtctcaacaaaatcttaacattgacaaagatacaaccttgcttgcttcggctaaaaaagcaaagacgtctgtgtcaaagaaggactctcagaagaatgataaagatgtgaagagattacaaaaacacattcgcgctcttttgcaagagcgagggactcaggacaaacggatccaggacatggaatctgaattggagaagacagaagcaaagctcaatgcagcagtcagagagaaaacatctctctctgcgagtaatgcttcactggaaaaacggcttactgaattaaccagagccaacgagctactaaaggctaagttttctgaagatggtcaccaaaagaatatgagagctctaagcctggaattgatgaaactcagaaataagagagagacaaagatgaggagtatgatggtcaaacaggaaggcatggagctgaagctgcaggccactcagaaggacctcacggagtctaagggaaaaatagtccagctggagggaaagcttgtttcaatagagaaagaaaagatcgatgaaaaatgtgaaacagaaaaactcttagaatacatccaagaaattagctgtgcatctgatcaagtggaaaaatgcaaagtagatattgcccagttagaagaagatttgaaagagaaggatcgtgagattttaagtcttaagcagtctcttgaggaaaacattacattttctaagcaaatagaagacctgactgttaaatgccagctacttgaaacagaaagagacaaccttgtcagcaaggatagagaaagggctgaaactctcagtgctgagatgcagatcctgacagagaggctggctctggaaaggcaagaatatgaaaagctgcaacaaaaagaattgcaaagccagtcacttctgcagcaagagaaggaactgtctgctcgtctgcagcagcagctctgctctttccaagaggaaatgacttctgagaagaacgtctttaaagaagagctaaagctcgccctggctgagttggatgcggtccagcagaaggaggagcagagtgaaaggctggttaaacagctggaagaggaaaggaagtcaactgcagaacaactgacgcggctggacaacctgctgagagagaaagaagttgaactggagaaacatattgctgctcacgcccaagccatcttgattgcacaagagaagtataatgacacagcacagagtctgagggacgtcactgctcagttggaaagtgtgcaagagaagtataatgacacagcacagagtctgagggacgtcactgctcagttggaaagtgagcaagagaagtacaatgacacagcacagagtctgagggacgtcactgctcagttggaaagtgagcaagagaagtacaatgacacagcacagagtctgagggacgtcactgctcagttggaaagtgtgcaagagaagtacaatgacacagcacagagtctgagggacgtcactgctcagttggaaagctataagtcatcaacacttaaagaaatagaagatcttaaactggagaatttgactctacaagaaaaagtagctatggctgaaaaaagtgtagaagatgttcaacagcagatattgacagctgagagcacaaatcaagaatatgcaaggatggttcaagatttgcagaacagatcaaccttaaaagaagaagaaattaaagaaatcacatcttcatttcttgagaaaataactgatttgaaaaatcaactcagacaacaagatgaagactttaggaagcagctggaagagaaaggaaaaagaacagcagagaaagaaaatgtaatgacagaattaaccatggaaattaataaatggcgtctcctatatgatgaactatatgaaaaaactaaaccttttcagcaacaactggatgcctttgaagccgagaaacaggcattgttgaatgaacatggtgcaactcaggagcagctaaataaaatcagagactcctatgcacagctacttggtcaccagaacctaaagcaaaaaatcaaacatgttgtgaaattgaaagatgaaaatagccaactcaaatcggaggtgtcaaaactccgatctcagcttgttaaaaggaaacaaaatgagctcagacttcagggagaattagataaagctctgggcatcagacactttgacccttccaaggctttttgtcatgcatctaaggagaattttactccattaaaagaaggcaacccaaactgctgctgagttcagatgcaacttcaagaatcatggaagtatacgtctgaaatacttgttgaagattatttttctt

SEQ ID NO: 6 - DNA, 2286 bp, Rattus norvegicus
ttgcatctggacccaggcgctagaatgtccttccctaaggcgcccctgaaaagattcaatgacccttcgggttgtgctccatctccaggtgcttacgatgttaaaacttcagaatcaactaaaggaccagtgtcttttcagaaatcacaaagatttaaaaaccaaagagagtctcaacaaaatcttaacattgacaaagatacgaccttgcttgcttctgctaaaaaagcaaagacgttggtgtcaaagaaggagtctcagaagaatgataaagatgtgaagagattagaaaaagagattcatgttcttttgcaagaacgagggactcaggacaaacggatccaggacatggagtctgagttggagaatacagaagccaatctaaatgcaccagtcacagagaaaccatctctgtctgccaataatgcttcactggaaaaacggcttactgagctgaccagagccaacgagctcctaaagtctaagttttctgaagatgctcaccaaaagaatatgagaactctaagcctggaattgatgaaactgagaaataagagagagacaaagatgaggagcatgatggctaaacaggaaggcatggagctgaagctgcaggccactcagaaggacctcattgagtctaaggggaaaatagtacagctggagggaaaacttgtttcaatagagaaagaaaagatcgatgaaaaatctgaaacagaaaaactcttagaatacatagaagaaattagctgtgcatctgatcaagtggaaaaatacaaactagatattgcccagttagaagaagatttgaaagagaaggatcgtgagattttatgccttaagcaatctcttgaggaaaaggtttccttttctaagcaaatagaagacctgactgttaaatgtcagctgcttgaagcagaaagagatgatctcgtcagcaaggacagagaaagggctgaaagcctcagtgctgagatgcaggttctaacagagaagctgcttctagaaaggcaagaatatgaaaagctgcaacaaaatgaattgcaaagccagtcacttctgcagcaagaaaaggaactgtctgctcatcttcagcagcagctctgctcatttcaagaggaaatgacctccgagaggaatgtctttaaagaacagttgaagcttgccctggatgagctggatgccgtccagcagaaaaaggagcagagtgaaaagctggttaaacagctagaagaggaaacgaagtcaactgcggaacagctgaggcggctggatgatctgctgagagagaaagaaattgaactggagaaaagaaccgctgcacatgcccaggccactgtgattgcgcaagagaagtacagtgacacagcgcagactctgagagatgttactgctcagttagaaagctataagtcatcaacacttaaagaaatagaagatcttaaactggagaatttgactctacaagaaaaagtagccatggctgagaaaagggtagaagatgttcaacaacaaatattaacagctgagagcacaaatcaggaatatgcaaaggtggttcaagatttgcagaacagctcaacattgaaagaagcagaaattaaagaaatcacatcttcatatcttgagaaaataactgatttgcaaaatcaactccgacaacaaaatgaagactttaggaagcagctggaagaggaaggggcaaaaatgacagagaaagaaactgcagtgacagaattaaccatggaaattaataaatggcgtctcctatatgaagaactgtttgacaaaactaaaccttttcagcaacaactggatgccttcgaagcagagaagcaggcactgttgaatgaacatggtgcaactcaggagcagctaagtaaaatcagagactcctacgcacagctgcttggccaccagaatctgaagcaaaaaatcaaacatgtcgtgaaattgaaagatgaaaatagccagctcaaatcggaggtgtcaaaactccgatctcagcttgctaaaaggaaacaaaatgagctcagacttcagggagaattagataaagctctgggcatcaggcactttgacccttctaaggctttttgccatgaatctaaggagaatgtgaccctcaagactccattgaaagaaggcaacccgaactgctgctgagtcagactgcagggaccgtggaagtggacgtccaagatacttgctgaagattgttctcttcattattcttgatattatgtttatagtatatattatataatgtatttaatttctactgcc

SEQ ID NO: 7 - protein, 8 aa, Artificial Sequence
  HA-binding peptide
KQKKHXXK

SEQ ID NO: 8 - protein, 9 aa, Artificial Sequence
  HA-binding peptide
KIKHXXKXK

SEQ ID NO: 9 - protein, 10 aa, Artificial Sequence
  HA-binding peptide
KLRSQLXKLR

SEQ ID NO: 10 - protein, 10 aa, Artificial Sequence
  HA-binding peptide
KLRSQLXKRK

SEQ ID NO: 11 - protein, 15 aa, Artificial Sequence
  HA-binding peptide
TMTRPHFHKRQLVLS

SEQ ID NO: 12 - protein, 15 aa, Artificial Sequence
  HA-binding peptide
STMMSRSHKTRSCHH

SEQ ID NO: 13 - protein, 14 aa, Artificial Sequence
  HA-binding peptide
STMMSRSHKTRSHH

SEQ ID NO: 14 - protein, 15 aa, Artificial Sequence
  HA-binding peptide
STMMSRSHKTRSHHV

(19) United States
(12) Patent Application Publication
(10) Pub. No.: US 2004/0037834 A1
(43) Pub. Date: Feb. 26, 2004

(54) Rhamm peptide conjugates

(76) Inventors: B. Michael R. Woloski, Charlottesville, VA (US); Ashley Martin Williams, Winnipe Manitoba Canada (CA); Terrance Jimmy Sereda, Winnipeg Manitoba Canada (CA); Deanna June Wiebe, Winnipeg Manitoba Canada (CA)

Correspondence Address: KNOBLE & YOSHIDA, EIGHT PENN CENTER, SUITE 1350, 1628 JOHN F KENNEDY BLVD, PHILADELPHIA, PA, 19103, US

(21) Appl. No.: 10/257,377

(22) PCT Filed: Apr. 20, 2001
(86) PCT No.: PCT/CA01/00533

(51) Int. Cl. A61K 39/395; A61K 48/00; A61K 38/17; C07K 16/46; C07K 14/705

(52) U.S. Cl. 424/178.1; 530/391.1; 514/12; 530/350; 435/69.1; 435/320.1; 435/326

(57) ABSTRACT

The present invention provides protein conjugates having a glucose-aminoglycan-targeting domain conjugated directly or indirectly to a therapeutically useful protein via chemical or peptidyl linkage. The protein conjugates selectively target certain tissues and organs and are useful for treating or preventing various physiological and pathological conditions. Methods of their use and preparation are described.

### FIELD OF THE INVENTION

The present invention relates to drug-polypeptide conjugates and methods of their use in treating or preventing various physiological and pathological conditions. Specifically, the conjugates described herein comprise an effective amount of one or more therapeutic agent(s) conjugated to one or more peptide(s) or polypeptide(s) capable of binding hyaluronan. The invention also relates to nucleic acid molecules encoding such conjugates and various processes for producing such conjugates.

### BACKGROUND OF THE INVENTION

Hyaluronan (hyaluronic acid or HA) is a coiled, negatively charged, ubiquitous glucosaminoglycan comprised of repeating disaccharide units of N-acetylglucosamine and glucuronic acid (Ogston and Stanier, 1951, 1953). Circulating HA is primarily of peripheral tissue origin, and enters the blood through the lymph (Laurent et al., 1986). In addition to its known physicochemical functions, such as lubrication in synovial fluid in joints, HA also performs macrostructural functions in skin, cartilage, and brain tissue (Reed et al., 1988). HA is produced and secreted locally by chondrocytes (Asari et al., 1994, Noonan et al., 1996), and is also found in connective tissue and cartilage, comprising an integral component of the extracellular matrix, which regulates proliferation, differentiation, and maturation of cells (Oguri et al., 1987). Specifically, HA exerts its control of hematopoiesis at the early progenitor stage prior to a commitment to either myeloid or lymphoid lineage. In addition, HA enhances myelopoiesis and lymphopoiesis by induction of interleukin production (Khaldoyanidi et al., 1999) Concentration of hyaluronic acid is highest in embryonic tissue. In loose connective tissue, it is found at a concentration of 1-3 mg/cm³, and 0.02-1 mg/cm3 in vitreous humor. Due to the large solution domain of hyaluronic acid, at concentrations above 1 mg/cm3 the molecules will become entangled, forming a polymeric network (Comper and Laurent, 1978). This distribution of endogenous HA should be distinguished from the distribution of exogenously administered HA, which is subject to first pass metabolism by the liver, and does not accumulate significantly in bone, connective tissue, skin, cartilage, joints, or brain tissue (Fraser et al., 1981, 1985).

Synthesis of HA has been associated with the morphogenesis of many tissues, with wound repair, tumour invasion and cellular immune function (Boudreau et al., 1991; Iozzo, 1985; Pauli et al., 1983; Toole, 1982; Toole et al., 1989; Turley, 1984, 1992; Weigel et al., 1986, 1989). Increased levels of HA have been documented in rheumatoid arthritis, scleroderma, fibrosis of the liver, and in cancer cells (Ichida et al., 1996, Laurent et al., 1986). The underlying mechanism of action at the cellular level is believed to involve the ability of HA to elicit receptor-mediated alterations of cell motility.

High affinity HA receptors have been identified and characterized on a variety of cell types and these are namely the receptor for HA mediated mobility (or RHAMM) (Turley, E. A. et al., 1991; Hardwick, C. et al., 1992; Yang, B. et al., 1993), intercellular adhesion molecule-1 (or ICAM-1) (McCourt, P. A. G. et al., 1994) and CD44 (Underhill, C. B. et al., 1987; Stamenkovic, I. et al., 1989; Aruffo, A. et al., 1990; Lesley, J. et al., 1990; Miyake, K. et al., 1990). Both RHAMM and CD44 have been shown to be associated with cell locomotion, cell proliferation and differentiation. Other HA-binding proteins in the extracellular milieu have also been identified and they are namely link protein, aggrecan, versican, GHAP, collagen type VI and TSG-6.

RHAMM was one of the first HA receptors to be isolated and characterized at the biochemical and molecular levels. It is an N-linked glycoprotein that binds HA with high affinity (Kd: 1 nM) and specificity. Several isoforms of RHAMM with different subcellular distribution have been identified. iRHAMM and pRHAMM are isoforms found intracellularly and on the plasma membrane, respectively, and sRHAMM is the form of a secreted protein. The molecular structure of the various RHAMM isoforms may be differentially regulated by phosphorylation and/or glycosylation status. The precise roles of the RHAMM isoforms have not been fully elucidated, but it is believed that pRHAMM and sRHAMM elicit opposite activities and the net functional behaviour of a HA-RHAMM interaction depends at least in part on the balance of pRHAMM versus sRHAMM expressed by the cells involved.

A full-length murine RHAMM cDNA has been cloned successfully from a GT11 3T3 cDNA expression library (Hardwick, C. et al., 1992). Immunoblot analyses of cell lysates using antibodies to peptides encoded in the cDNA reacted specifically reacted with RHAMM protein. Using a fragment of the clone DNA sequence, a mouse fibroblast genomic library was screened to clone the genomic RHAMM gene which spans at least 20 kb and comprises 14 exons ranging in size from 75 to 1099 bp (Entwistle, et al., 1995).

Similarly, a human RHAMM cDNA clone was also isolated successfully by a combination of screening a human breast cDNA expression library with the murine RHAMM cDNA as well as 5′ RACE and reverse transcription-polymerase chain reaction using messenger RNA from the human breast cell line MCF-10A (Wang, et al., 1996). The full-length human RHAMM cDNA encodes for a 725 amino acid protein and shares a 85% homology with the the murine transcripts, RHAMM v4. More importantly, the HA binding motif B1-An-B2 which is shown to be critical for the signaling capability of RHAMM is 100% conserved between human and mouse.

Turley in PCT application PCT/CA98/00448 published as WO 98/52590 also described the interposing of a hyaluronan binding motif between a disease modifier and exogenous hyaluronan. The exogenous hyaluronan described in this patent publication is claimed to be less than 750,000 daltons in molecular weight.

Colony stimulating factors (CSF's) are natural hematopoietic growth factors that regulate the production and functional activity of red blood cells, white blood cells, and platelets. The most important CSF's to date are erythropoietin (EPO), granulocyte-colony stimulating factor (G-CSF), granulocyte-macrophage-colony stimulating factor (GM-CSF), macrophage-colony stimulating factor (M-CSF), interleukins (IL's), and stem cell growth factor (SCF). The continuous presence of CSF's in the concentration range of 1-100 pM is required for the proliferation of hematopoietic progenitor cells. Accordingly, CSF's can be used to restore hematopoietic dysfunction, to augment host defense against infection, and to stimulate the proliferation of specific cells.

GM-CSF and G-CSF stimulate the production of granulocytes and/or macrophages, extend the life expectancy of mature white cells, and potentiate their biological activity. These white cells form the basis of the body's immune system, which may be compromised by events ranging from chemotherapy or infection to idiopathic neutropenia. In addition to reducing the duration of hospitalization, intensity of hospitalized treatment, and antibiotic usage among cancer patients, both G-CSF and GM-CSF permit higher, more frequent doses of chemotherapy and improve overall prognosis. Approved and investigational therapeutic indications for GM-CSF include the treatment of the following disorders: (1) myeloid reconstitution in autologous bone marrow transplantation in non-Hodgkin's lymphoma, acute lymphocytic leukemia, and Hodgkin's Disease; (2) allogenic bone marrow transplantation failure or engraftment delay; (3) acute myelogenous leukemia following high dose chemotherapy; (4) peripheral blood progenitor cell mobilization and transplantation; (5) treatment of chemotherapy-induced neutropenia (approved in Europe); (6) to increase WBC counts in myelodysplastic syndrome (approved in Europe); (7) to increase WBC counts in AIDS patients receiving AZT treatment; (8) treatment of leukopenia secondary to chemotherapy; (9) treatment of neutropenia in aplastic anemia (approved in Finland); (10) prolong survival of patients with advanced melanoma (phase II/III); (11) treatment of neutropenia due to hairy cell leukemia; (12) chronic lymphocytic leukemia therapy; (13) treatment of neutropenia of Felty's syndrome; (14) thermal injuries (>40% full/partial burns); (15) drug-induced neutropenia (aside from AZT and cancer chemotherapy); and (16) ganciclovir-induced neutropenia is approved in Europe.

Similarly, approved and investigational therapeutic indications for G-CSF include the treatment of the following disorders: (1) to decrease the incidence of infection (febrile neutropenia) in patients with non-myeloid malignancies receiving myelosuppressive cancer chemotherapy and radiation therapy; (2) treatment of severe chronic neutropenia (SCN) including congenital, cyclical, or idiopathic neutropenia; (3) use in allogenic/autologous bone marrow transplantation and peripheral blood progenitor cell (PBPC) transplantation; (4) AIDS-associated neutropenia—approved in U.K; (5) acute myelogenous leukemia; (6) dose escalation of chemotherapy—Myelodysplastic syndrome; (7) severe community acquired pneumonia; (8) intra-abdominal infections; (9) pneumonia/sepsis; (10) invasive fungal infection; (11) liver transplantation; (12) neonatal infection; and (13) head injury.

Erythropioetin (EPO) is endogenously produced by the juxtatubular cells in the kidney, and to a smaller extent by the liver. The final product is a heavily glycosylated protein with 166 amino acid residues and a molecular weight of approximately 34,000D. Although EPO is not the only growth factor responsible for erythropoiesis, it is the most important stimulant for red blood cell production, by regulating proliferation of committed progenitors, maturation of erythroblasts, and release of reticulocytes into the circulation. EPO is currently approved in Canada and the USA, and is under investigation, for the treatment of conditions as follows: (1) anemia associated with chronic or progressive renal failure to improve quality of life and maintain hematocrit levels; (2) anemia associated with symptomatic HIV infection (AIDS); (3) anemia associated with non-myeloid neoplastic diseases that may be exacerbated by chemotherapy, to stimulate erythropoiesis and reduce the need for transfusion following chemotherapy; (4) autologous transfusion in patients undergoing elective surgery; (5) anemia in premature infants; (6) anemia associated with other chronic diseases (e.g. rheumatoid arthritis); (7) beta-thalassemia or sickle cell anemia; and (8) orthostatic hypertension.

Human growth hormone (hGH) is an adenohypophyseal pituitary hormonal polypeptide of 191 amino acids with an approximate molecular weight of 22,000D. The principal form of the molecule is a single chain with two intrachain disulfide linkages and no covalently bound carbohydrates. The primary function of hGH in humans is to stimulate organ and tissue growth through positive nitrogen balance and other tissue ionic constituents such as calcium, magnesium, sodium, potassium, and phosphate. hGH binds to cell-surface receptors throughout the body to enhance amino acid uptake and protein synthesis by body cells while reducing protein catabolism. hGH also exerts a number of complex effects on human lipid and carbohydrate metabolism. Briefly, hGH may be considered to be diabetogenic and switches the bodily source of fuel from carbohydrate to fat. The intracellular effects of hGH binding are primarily mediated by insulin-like growth factor 1 (IGF-1) as well as IGF-2. Long-term administration of the hormone has been documented to induce lipid mobilization, reduce body fat stores, and increase plasma fatty acids.

hGH is currently approved and is under investigation for therapeutic use in the conditions as follows: (1) growth hormone insufficiency; (2) growth insufficiency in Turner's syndrome; (3) chronic renal insufficiency; (4) AIDS related wasting; (5) adult growth insufficiency; (6) patients with osteoporosis may benefit from the calcium; (7) retention and osteogenic effects of hGH therapy; (8) muscle wasting associated with cancer; (9) patients of short stature with Down's syndrome, nephrotic cystinosis, neural tube defects, intrauterine growth retardation, and cancer radiation treatment; (10) adjunct therapy in the treatment of catabolic conditions in burn injuries; (11) sensitization of the ovary to gonadotrophins in infertility; (12) achondroplasia; and (13) veterinary and agricultural purposes.

Interferons are potent cytokines that stimulate complex antiviral, immunomodulatory, and antiproliferative effects in response to viral infection and other biological inducers. There are three classes of interferons, namely alpha (leukocyte), beta (fibroblast), and gamma (immune), which bind their respective cell-surface receptors to enhance macrophage function, induce production of immunomodulatory proteins and cytokine receptors, and prevent viral penetration, reproduction, and protein synthesis.

Approved and investigational indications for alpha-interferon are as follows: (1) hairy-cell leukemia; (2) AIDS-related Kaposi's syndrome; (3) chronic myelogenous leukemia; (4) basal cell carcinoma; (5) multiple myeloma; (6) hepatitis; (7) juvenile laryngeal papillomatosis; (8) condylomata acuminata; (9) genital herpes; (10) cancer of the head and neck; (11) combination therapy for AIDS; (12) combination therapy in squamous cell cancer of the cervix and skin; (13) ovarian carcinoma; (14) cervical carcinoma; (15) bladder carcinoma; (16) primary malignant brain tumor; (17) renal cell carcinoma; (18) malignant melanoma; (19) esophageal carcinoma; (20) colon cancer; and (21) multiple sclerosis.

Parathyroid hormone (PTH) is an 84 amino acid polypeptide chain with a molecular weight of approximately 9500 D. The primary role of PTH is to maintain calcium concentration in the extracellular fluid, which is accomplished mainly through the activation of osteoclasts to release calcium from bone. In addition, PTH increases calcium reabsorption from the intestinal tract and kidneys, and regulates the excretion of calcium in feces, sweat, and milk. Teriparatide, synthetic PTH available under the name Parathar, is used only as a diagnostic to differentiate between pseudohypoparathyroidism and hypoparathyroidism. Treatment of these diseases is accomplished with calcium and/or vitamin D, due to greater safety with these agents. Adverse effects following teriparatide treatment include constipation, headache, loss of appetite, muscle weakness, abdominal cramping, diarrhea, nausea, tingling in the hands and feet, and urge for bowel movement.

Interleukins (IL's) are multifunctional cytokines which are produced by and act on the lymphopoietic system. Over 15 interleukins have been identified and are under investigation for the treatment of various pathologies, primarily cancer (Dorland's Illustrated Medical dictionary).

Brief Description of Each are Set out Below:

IL-1 (lymphocyte-activating factor), is produced by macrophages, and induces IL-2 release from activated T cells. In the vasculature, epithelial cells produce IL-1 in order to stimulate fibroblast proliferation and enzyme and prostaglandin release.

IL-2 (T-cell growth factor) is produced by T cells following stimulation by IL-1. IL-2 stimulates T cell proliferation and production of interferon-g, and is used therapeutically in the treatment of solid malignant tumors.

IL-3 (multi-CSF) is produced by activated T cells, and stimulates colony formation of hematopoietic cell lines as well as influencing eosinophil and basophil function, and resulting in pulmonary macrophage proliferation. In addition to its direct effects on hematopoiesis, IL-3 acts synergistically with GM-CSF to increase blood neutrophils, eosinophils, and monocytes, and with EPO to expand the erythrocyte burst forming unit compartment and erythrocyte colony forming unit.

IL-4 (B-lymphocyte stimulating factor I) is released from activated T cells. IL-4 promotes maturation and growth of T cells and mast cells and regulates B cell function. This regulation includes proliferation, antigen presentation, and immunoglobulin production.

IL-5 is also produced by activated T cells to promote differentiation of B cells and eosinophils and increases production of IgA by B cells.

IL-6 is released from macrophages, fibroblasts, and activated T cells to stimulate thymocyte and B cell differentiation and immunoglobulin production.

IL-7 is produced by mesangial and epithelial stromal cells to promote early stage differentiation of lymphocytes.

IL-8 is released by monocytes and endothelial cells as a neutrophil chemotactic and activator.

IL-9 is a growth factor that is released from T cells and macrophages in vitro.

IL-10 inhibits the production of cytokines, increases B cell viability, and induces major histocompatability II gene expression.

Several other interleukins have been identified, and await specific characterization. Specific therapeutic uses of interleukins include (Harrison's Principles of Internal Medicine): (1) IL-1, 3, and 6 improve blood counts in some patients with aplastic anemia; (2) IL-1 improves platelet and granulocyte recovery following chemotherapy; (3) IL-2 is approved for the treatment of cancer, IL 4, IL-6, IL-12 are currently under investigation for this indication (University of Pittsburgh Cancer Institute); (4) clinical trials of IL-2 and IL-12 to stimulate T cells and natural killer cells in AIDS patients are underway; (5) IL-8 has been shown to induce de novo angiogenesis in a rat model (Norrby, et al., 1996); (6) IL-10 decreases the severity of psoriasis (Asadullah et al., 1999); (7) IL 11 has been approved for the treatment of chemotherapy-induced thrombocytopenia (Schwertschlag et al., 1999); (8) IL-11 exhibits anti-inflammatory effects in animal models of inflammatory bowel disease, inflammatory skin disease, autoimmune joint disease, and various infection-endotoxemia syndromes; IL-11 is also under investigation in the treatment of Crohn's disease, psoriasis, and rheumatoid arthritis (Schwertschlag et al., 1999); and (9) IL-12 may help control mycobacterium avium infection (NIH, 1998).

### SUMMARY OF INVENTION

The present inventors have by their invention developed conjugates which specifically target tissues and organs enriched in endogenous HA such as the lymph nodes, bone marrow, skin and muscle. Accordingly, these conjugates can exert focussed therapeutic action(s) directly on the site of the disorder thereby allowing lower dosages and/or frequency of drug administration required to achieve equivalent therapeutic efficacy as the conventional dosages of the corresponding unconjugated therapeutic protein alone. Lower therapeutic dosages required also translates into lower immunogenicity of the conjugated protein as compared to the native protein. As a result, conjugates of the present invention would improve patient compliance and reduce direct and indirect costs associated with the drug substance and its administration. Accordingly, in its broad aspsect the present invention provides a conjugate with one or more HA-binding protein(s) and/or polypeptide(s) conjugated to one or more therapeutic agent(s).

The critical distinction between the conjugates of the present invention from the subject matter set forth in the Turley PCT application PCT/CA98/00448 mentioned above is that the conjugates of the present invention are not and should not be interposed in any manner with exogenous hyaluronan. The inclusion of hyaluronan in the conjugates of the present invention would dramatically alter their targeting profile thereby obliterating their ability to target to the tissues requiring the desired treatment.

For example, exogenously administered hyaluronan is entrapped in metabolic organs such as the liver and the interposing of exogenous hyaluronan to the conjugates of the present invention would facilitate the degradation of the conjugate and eliminate the desired therapeutic outcomes of the conjugates. By contrast, conjugates of the present invention when administered without exogenous hyaluronan would target different tissues that are naturally rich in endogenous HA where the desired therapeutic site of action is required.

One aspect of the present invention provides conjugated sequence(s) of amino acids each having an amino acid sequence encoding an HA-binding protein linked to an effective amount of one or more therapeutic agent(s). Non-limiting examples of such HA-binding protein include receptor for hyaluronan-mediated motility (RHAMM) or a variant or fragment thereof capable of binding HA and anti-HA antibodies.

A second aspect of the present invention provides conjugated polypeptides each having an amino acid sequence comprising a peptide having HA-binding affinity; linked to an effective amount of therapeutic agent(s). Non-limiting examples of such therapeutic agents include CSF, EPO, hGH, interferons, PTH and ILs.

Another aspect of the present invention provides isolated and purified nucleic acid sequences encoding an HA-binding peptide in sequence with a therapeutic agent, with or without the presence of an intervening amino acid sequence.

A further aspect of the present invention provides a method for preparing the conjugate molecules of the present invention.

In one embodiment, the preparation of the conjugate molecule comprises the following steps: (i) inserting an appropriate nucleotide sequence (encoding a HA binding protein or peptide) directly or indirectly linked to a nucleotide sequence (encoding a therapeutic protein) into a suitable vector; (ii) expressing the vector in an acceptable host; and (iii) purifying said conjugate molecule from said host or expression medium. In a further embodiment, the preparation of the conjugate molecule comprises the acquisition of the HA binding protein or peptide and therapeutic agent, and the linkage of these domains by one of various chemical and enzymatic methods known to persons skilled in the art.

Also provided is a process for preparing a pharmaceutical for treating an animal in need of treatment comprising the steps of preparing a purified conjugate of the present invention and suspending the conjugate molecule in a pharmaceutically acceptable carrier, diluent or excipient.

A further aspect of the present invention provides a pharmaceutical composition comprising an HA binding protein or peptide directly or indirectly conjugated to a therapeutic agent suspended in a pharmaceutically acceptable carrier, diluent, or excipient.

In yet another aspect, the present invention provides a method for altering in vivo the distribution of a therapeutic agent. According to one embodiment the distribution is altered by conjugating the therapeutic agent to one or more HA-binding protein(s) or peptide(s), administering the conjugate to the animal where the conjugate molecule will distribute primarily in tissues and organs containing high levels of endogenous HA. The present invention also provides a method of treating a mammal with a disorder where the diseased tissue/organ contains high levels of HA by administering the conjugate molecules of the invention to said mammal.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present inventors found that conjugated therapeutic agents having therapeutic properties when conjugated to a sequence of amino acids which bind HA, surprisingly, have an enhanced ability to target certain diseased tissues and organs enriched in HA. As discussed above, this allows for the use, where appropriate, of lower, safer, dosages of these conjugates as compared to the conventional dosage requirements for the unconjugated corresponding therapeutic agent.

The term “HA-binding Protein” as used herein means any polypeptide or protein that can bind hyaluronic acid (or HA). Preferred embodiments of the HA-binding proteins are isolated natural receptors that can bind HA (RHAMM) or antibodies raised against or specifically binds to HA (e.g. Formula I, II, or III). The term HA-binding polypeptide or protein also includes fragments, analogs and derivatives of RHAMM or anti-HA antibodies, which maintain the ability to bind HA.

The term “HA-binding peptide” as used herein means any peptide that can bind hyaluronic acid (or HA). Preferred embodiments of the HA-binding peptides are of the Formula I, II, or III as defined herein. The term HA-binding peptide includes fragments, analogs and derivatives of the peptides, which maintain the ability to bind HA. Collectively, the HA binding peptides defined herein are referred to as RHAMM peptides. Preferably, RHAMM peptides consist of amino acids in the levorotatory (L) form, which corresponds to the configuration of amino acids in nature.

The term “analog” includes any peptide having an amino acid residue sequence substantially identical to the sequence of the HA binding peptides shown in Formula I, II, or III shown herein in which one or more residues have been conservatively substituted with a functionally similar residue and which displays the ability to mimic a HA-binding peptide. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such polypeptide displays the requisite activity.

The term “directly” in the context of protein or nucleotide conjugation refers to the linkage of one amino acid or nucleic acid sequence to another without any additional intervening amino acids, nucleotides, or other molecules.

The term “animal” as used herein includes all members of the animal kingdom, including humans. Preferably, the animal to be treated is a human.

The term “indirectly conjugated” refers to the linkage of two amino acid or nucleic acid sequences by the insertion of amino acid residue(s), nucleic add(s), or other molecule(s) such as a chemical polymer between the two sequences to be linked.

The term “derivative” as used herein refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides that contain one or more naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine; and ornithine may be substituted for lysine. HA-binding peptides as provided herein also include any peptide having one or more additions and/or deletions or residues relative to the sequence of a polypeptide whose sequence is shown herein, so long as the requisite activity is maintained or increased.

The term “fragment” refers to any subject peptide having an amino acid residue sequence shorter than that of a peptide whose amino acid residue sequence is shown or is being referred to.

Conjugated Proteins

The present invention provides conjugated proteins or polypeptides each having one or more HA-binding protein(s) or HA-binding peptide(s) linked directly or indirectly to one or more therapeutic agent(s). For greater certainty, one therapeutic agent may be conjugated directly or indirectly to one or more HA-binding protein(s) and/or HA-binding peptide(s), while similarly, one or more therapeutic agent(s) may be conjugated directly or indirectly to an HA-binding protein and/or HA-binding peptide. Without limiting the above, the present inventors also contemplate conjugation of more than one of the same or different therapeutic agents to more than one of the same or different HA-binding proteins and/or HA-binding peptides to form a polymeric scaffold of therapeutic agents, HA-binding proteins and/or HA-binding peptides.

The selection of therapeutic agents, HA-binding proteins, and HA-binding peptides for conjugation and the stoichiometric ratios of each component to be used would depend on the physicochemical characteristics of the therapeutic agent(s) as well as the pharmacodynamic and pharmacokinetic requirements for the conjugate molecule on a case-by-case basis. In one embodiment, a therapeutic agent is linked directly or indirectly to an HA-binding protein which is a polypeptide comprising an amino acid sequence provided herein as SEQ ID NO. 1, SEQ ID NO. 2 or SEQ ID NO. 3, or a fragment, analog or derivative thereof which maintains the ability to bind HA. In another embodiment, a therapeutic agent is linked directly or indirectly to an HA-binding protein and may be a polyclonal or monoclonal antibody with binding affinity for HA.

In the event that the therapeutic agent is a polypeptide, the RHAMM-therapeutic protein conjugate may be prepared preferably using conventional recombinant DNA methodology. Alternatively, they may be produced separately as two sequences and subsequently conjugated by conventional chemical and enzymatic techniques well known to those skilled in the art of coupling of amino acids and peptides.

A second aspect of the present invention is concerned with conjugated polypeptides each comprising an HA-binding peptide sequence linked directly or indirectly to a therapeutic agent. The HA-binding peptide or the entire HA-binding peptide therapeutic protein conjugate may be prepared by chemical synthesis using techniques well known to those skilled in the art of chemistry of proteins, such as solid phase synthesis (Merrifield, 1964) or synthesis in homogenous solution (Houbenweyl, 1987).

In an embodiment, the HA-binding peptide has an amino acid sequence comprising a structure of B¹-Aₙ-B² wherein B¹ and B² are the same or different basic amino acid residues and An is an amino acid sequence containing seven or eight amino acid resides which are the same or different and are neutral or basic amino acids. Preferably, the HA-binding peptide comprises an amino acid sequence selected from the group consisting of KQKKHXXK, KIKHXXKUK, KLRsQLXKIR and KLRSQLXKRK wherein each X is independently selected from V or D (SEQ. ID. NOS.: 7 to 10).

In another embodiment, the HA binding peptide that can be conjugated directly or indirectly to a therapeutic protein has an amino acid sequence comprising of the following Formula I:

X₁—X₂—X₁—X₃—X₄—X₃—X₄—X₃—X₃—X₃—X₅—X₆—X₆—X₆—X₁

wherein:

each X₁ is independently selected from a hydroxy amino acid residue;

each X₂ is independently selected from a sulfur containing amino acid residue;

each X₃ is independently selected from a basic amino acid residue;

each X₄ is independently selected from an imino or aromatic amino acid residue;

each X₅ is independently selected from a dicarboxylic acid amino acid residue; and

each X₆ is independently selected from an aliphatic amino acid residue;

and preferably:

each X₁ is independently selected from threonine or serine;

each X₂ is independently selected from methionine or cysteine;

each X₃ is independently selected from arginine, lysine or histidine;

each X₄ is independently selected from proline, phenylalanine or tryptophan;

each X₅ is independently selected from asparagine or glutamine; and

each X₆ is independently selected from leucine, isoleucine, valine or alanine.

A preferred amino acid sequence of this Formula I is TMTRPHFHKRQLVLS (SEQ. ID. NO.: 11).

In a further embodiment, the HA binding peptide that can be conjugated directly or indirectly to a therapeutic protein has an amino acid sequence comprising of the following Formula II:

Y₁—Y₁—Y₂—Y₂—Y₁—Y₃—Y₁—Y₃—Y₃—Y₁—Y₃—Y₁—Y₂—Y₃—Y₃

wherein:

each Y₁ is independently selected from a hydroxy amino acid residue;

each Y₂ is independently selected from a sulfur containing amino acid residue; and

each Y₃ is independently selected from a basic amino acid residue;

and preferably:

each Y₁ is independently selected from serine or threonine;

each Y₂ is independently selected from methionine or cysteine; and

each Y₃ is independently selected from arginine, lysine or histidine.

A preferred amino acid sequence of this Formula II is STMMSRSHKTRSCHH (SEQ. ID. NO.: 12).

In another embodiment, the HA binding peptide that can be conjugated directly or indirectly to a therapeutic protein has an amino acid sequence comprising of the following Formula III:

Z₁-Z₁-Z₂-Z₂-Z₁-Z₃-Z₁-Z₃-Z₃-Z₁-Z₃-Z₁-Z₃-Z₃

wherein:

each Z₁ is independently selected from a hydroxy amino acid residue;

each Z₂ is independently selected from a sulfur containing amino acid residue; and

each Z₃ is independently selected from a basic amino acid residue, and fragments;

and preferably,

each Z₁ is independently selected from serine or threonine;

each Z₂ is independently selected from methionine or cysteine; and

each Z₃ is independently selected from arginine, lysine or histidine,

and fragments, analogs or derivatives of the peptide which bind HA.

A preferred amino acid sequence of this Formula III is STMMSRSHKFRSHH (SEQ. ID. NO.: 13). This SEQ. ID. NO.: 13 may optionally contain a valine residue at the C-terminal and have the following sequence: STMMSRSHKTRSHHV (SEQ. ID. NO.: 14).

As stated above, the present invention concerns RHAMM peptides and their conjugation to a therapeutic protein. More than half of endogenously produced HA is distributed in skin tissue, and approximately a quarter is distributed in skeletal tissue. Accordingly, a pathological condition or disease condition occuring in these tissues will benefit as therapeutic proteins useful in treating such conditions are targetted to these tissues by a conjugate of the present invention. The therapeutic protein may be chosen from the group comprising a colony stimulating factor (G-CSF, GM-CSF or M-CSF), erythropoietin, growth hormone, parathyroid hormone, an interferon, an interleukin, an antibody, an immunoadhesin, or any functional fragment thereof.

The conjugates of the present invention allow the specific targeting of the therapeutic agent to tissues in which there is a high concentration of HA. An additional feature of the conjugate molecules is an increased half-life and potency, resulting in prolonged circulation of the molecule, efficient distribution into the target tissues, and increased bioavailability. Any therapeutic agent, protein or polypeptide that would benefit from direction to tissues with a high concentration of HA can be conjugated to RHAMM peptide under the present invention.

An embodiment of the present invention is the conjugation of a therapeutic protein to an HA-binding protein or HA-binding peptide.

A further embodiment of the present invention is the conjugation of a vaccine or antibody to an HA-binding protein or HA-binding peptide. A vaccine may be conjugated to RHAMM to deliver the vaccine directly to the lymph nodes, which is the site of antibody release into the circulation. As B and T cells mature, antibodies will recognize the vaccine antigen and induce an immune response, conferring protection against the antigen contained in the vaccine. The conjugation of an antibody (for example to a cancer antigen such as Her-2) to RHAMM could be used in such applications as cancer metastasis, as described above. As tumor cells migrate to the lymph nodes, the RHAMM-antibody conjugate will bind to the cells and induce an immune response, resulting in destruction of the migrating cancer cells.

Antibodies having specificity for a cell surface protein may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Monoclonal Antibodies in Cancer Therapy Allen R., Bliss, Inc., pages 77-96 (1985)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

As will now be discussed, conjugation techniques may include recombinant production of an amino acid sequence which may be translated into said conjugate molecule, or direct chemical ligation of the pep tide and protein, ligation of peptide and protein through intermediate linking molecules or sequences.

Methods of Preparing Conjugates

Recombinant Techniques

Isolated and purified conjugates of the present invention may be prepared using conventional recombinant DNA technology. The preparation of the conjugate molecule comprises the insertion of an appropriate nucleotide sequence (encoding a HA binding peptide directly or indirectly linked to a nucleotide sequence) into a suitable vector, its expression in an acceptable host, and its purification from said host.

The term “isolated and purified” as used herein refers to a nucleic acid substantially free of cellular material or culture medium when produced by recombinant DNA techniques, or chemical precursors, or other chemicals when chemically synthesized. An “isolated and purified” nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded.

The use of recombinant DNA methods to produce the conjugated proteins or unconjugated polypeptides of the present invention involves the insertion of nucleic acids the present invention into a known manner into an appropriate expression vector that ensures good expression of the protein. One embodiment provides isolated and purified nucleic acids having nucleotide sequences encoding an HA-binding peptides in sequence with a therapeutic protein may be synthesized, with or without the presence of an intervening amino acid sequence. The nucleic acid sequences of HA-binding peptides are essentially as those described in PCT patent application PCT/CA93/00158 published as WO 93/21312, Yang et al. (1993), and European patent publication EP 950,708. The nucleic acid sequences of example therapeutic proteins are essentially as those described in: (i) PCT patent application PCT/US84/02021 published as WO 85/02610 for erythropoietin; (ii) European patent publications EP 43,980 and EP 211,148 for alpha-interferon; (iii) U.S. Pat. Nos. 4,966,843 and 5,376,567 for interferon-beta 1 (NCBI Accession Nos. E00171 and CAA23796); (ii) European patent publications EP 220,574 and EP 536,520 for interferon-beta 2; (iv) U.S. Pat. Nos. 4,342,832 and 4,601,980 for human growth hormone; (v) PCT patent application PCT/US86/01708 published as WO 87/01132 for granulocyte-colony stimulating factor (G-CSF); (vi) U.S. Pat. Nos. 5,891,429 and 5,942,221 for granulocyte-macrophage-colony stimulating factor (GM-CSF); (vii) European patent publication EP 473,701 and U.S. Pat. No. 5,010,010 for (prepro)parathyroid hormone; (viii) European patent publications EP 165,654 and EP 259,160 for interleukin 1; (ix) European patent publications EP 118,977, EP 088,195 and EP 136,489 for interleukin 2; (x) PCT patent application PCT/US89/02599 published as WO 90/01039 and European patent publications EP 314,705 and EP 275,598 for interleukin 3; (xi) European patent publication EP 302,429 for interleukin 4; (xii) PCT patent application PCT/US89/05421 published as WO 90/06370 and European patent publication EP 316,319 for interleukin 6; (xiii) European patent publication EP 506,574 for interleukin 13; and (xiv) European patent publication EP 870,030 for interleukin 16.

The above nucleic acid sequences may each be obtained by chemical synthesis (joining of nucleotides) using conventional methods or by selective amplification of a coding region, using sets of degenerative primers or probes for selectively amplifying the coding region in a genomic or cDNA library. Appropriate primers may be selected from the nucleic acid sequence of HA-binding protein or peptide or a therapeutic protein. It is also possible to design synthetic oligonucleotide primers from the nucleotide sequences for use in PCR. The desired nucleic acid can then be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al., Biochemistry 18,5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which is operatively linked to the nucleic acid molecule. “Operatively linked” is intended to mean that the nucleic acid is linked to regulatory sequences in a manner that allows expression of the nucleic acid.

The invention therefore contemplates a recombinant expression vector of the invention containing a nucleic acid molecule of the invention, or a fragment thereof, and the necessary regulatory sequences for the transcription and translation of the inserted protein-sequence. Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native A and B chains and/or its flanking regions.

The recombinant expression vectors of the invention may also contain a selectable marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as b-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

The recombinant expression vectors may also contain genes that encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformant host cell. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

More particularly, bacterial host cells suitable for carrying out the present invention include E. coli, B. subtilis, Salmonella typhimurium, and various species within the genus' Pseudomonas, Streptomyces, and Staphylococcus, as well as many other bacterial species well known to one of ordinary skill in the art. Suitable bacterial expression vectors preferably comprise a promoter which functions in the host cell, one or more selectable phenotypic markers, and a bacterial origin of replication. Representative promoters include the b-lactamase (penicillinase) and lactose promoter system (see Chang et al., Nature 275:615 (1978)), the trp promoter (Nichols and Yanofsky, Meth in Enzymology 101:155, 1983) and the tac promoter (Russell et al., Gene 20: 231, 1982). Representative selectable markers include various antibiotic resistance markers such as the kanamycin or ampicillin resistance genes. Suitable expression vectors include but are not limited to bacteriophages such as lambda derivatives or plasmids such as pBR322 (see Bolivar et al., Gene 2:9S, (1977)), the pUC plasmids pUC18, pUC19, pUC118, pUC119 (see Messing, Meth in Enzymology 101:20-77,1983 and Vieira and Messing, Gene 19:259-268 (1982)), and pNH8A, pNH16a, pNH18a, and Bluescript M13 (Stratagene, La Jolla, Calif.). Typical fusion expression vectors which may be used are discussed above, e.g. pGEX (Amrad Corp., Melbourne, Australia), pMAL (New England Biolabs, Beverly, Mass.) and pRIT5 (Pharmacia, Piscataway, N.J.). Examples of inducible non-fusion expression vectors include pTrc (Amann et al., Gene 69:301-315 (1988)) and pET 11d (Studier et al., Gene Expression Technology: Methods in Enzymology 185, Academic Press, San Diego, Calif., 60-89 (1990)).

Yeast and fungi host cells suitable for carrying out the present invention include, but are not limited to Saccharomyces cerevisae, the genera Pichia or Kluyveromyces and various species of the genus Aspergillus. Examples of vectors for expression in yeast S. cerivisae include pYepSec1 (Baldari. et al., Embo J. 6:229-234 (1987)), pMFa (Kurjan and Herskowitz, Cell 30:933-943 (1982)), pJRY₈₈ (Schultz et al., Gene 54:113-123 (1987)), and pYES2 (Invitrogen Corporation, San Diego, Calif.). Protocols for the transformation of yeast and fungi are well known to those of ordinary skill in the art. (see Hinnen et al., Proc. Natl. Acad. Sci. USA 75:1929 (1978); Itoh et al., J. Bacteriology 153:163 (1983), and Cullen et al. (Bio/Technology 5:369 (1987)).

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the invention may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, pAS2022, pAS2023, and pAS2034).

Insect cells suitable for carrying out the present invention include cells and cell lines from Bombyx or Spodotera species. Baculovirus vectors available for expression of proteins in cultured insect cells (SF 9 cells) include the pAc series (Smith et al., Mol. Cell Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the invention are described in PCT/US/02442.

Alternatively, the proteins of the invention may also be expressed in non-human transgenic animals such as, rats, rabbits, sheep and pigs (see Hammer et al. (Nature 315:680-683 (1985)), Palmiter et al. (Science 222:809-814 (1983)), Brinster et al. (Proc. Natl. Acad. Sci. USA 82:4438-4442 (1985)), Palmiter and Brinster (Cell 41:343-345 (1985)) and U.S. Pat. No. 4,736,866).

Chemical and Other Synthetic Techniques

The preparation of the conjugate molecules of the present invention may also be achieved through linkage of the HA binding peptide and therapeutic protein by one of various chemical and enzymatic methods known to persons skilled in the art. Many large proteins, including bovine apocytochrome c, human endorphin analogs, beta-lipotropin, human pancreatic growth hormone-releasing factor, inhibin-92, ribonuclease A, bovine pancreatic trypsin inhibitor can be synthesized using various methods (Blake, 1986; Blake and Li, 1981, 1983; Blake et al., 1984, 1986; Cheng and Yamashiro, 1991; Jackson et al., 1994; Lu et al., 1998; Muir et al., 1994; Yamashiro and Li, 1988). Technologies relevant to the present invention include, but are not limited to: engineered protease-mediated ligation; coupling of minimally protected peptides in aqueous solution; backbone-engineered chemical ligation; native chemical ligation; and recombinant DNA technology. These examples are now further described.

Serine proteases have been used to conjugate synthetic, recombinant, or natural proteins and/or peptides with specific terminal sequences. Although the terminal sequence requirements for protease-mediated ligation are not absolute, enzymatic hydrolysis is thermodynamically favored over ligation. Therefore, reaction conditions (pH, temperature, and solvent concentration) may be altered to favor ligation, often at the expense of enzyme stability and substrate solubility (Homandberg, 1978, Wong, 1988). Recent adaptations to this technique have involved activation of the C-terminus of the N-terminal peptide to overcome the thermodynamic barrier. Such techniques are known to persons skilled in the art.

Various mutants of subtilisin have shown an increased ratio of ligase activity to hydrolysis activity, (Nakatsuka, 1987, Wu and Hilvert, 1989, Abrahmsen, 1991). Specifically, the mutant in which serine 221 is converted to cysteine and proline 225 is converted to alanine has been shown to increase the aminolysis to hydrolysis ratio of tetrapeptide esters and reduce steric crowding at the active site (Jackson et al., 1994). The conjugation procedure can be carried out site specifically in aqueous solution with high yield (Chang et al., 1994). This enzyme has been used in the construction of large proteins, including Ribonuclease A and semisynthesis of various other proteins (reviewed by Braisted et al., 1997).

Coupling of minimally protected peptides in aqueous solution will result in ligation of the C-terminus of a peptide to the N-terminus of a protein. In this method, the peptide is synthesized with a thiocarboxyl group (eg. thioglycine) at its C-terminus, while its amino groups and N-terminus are protected with citraconic anhydride. The C-terminal thiocarboxyl group is then activated by the addition of silver nitrate in the presence of N-hydroxysuccinimide, and the peptide is allowed to react with the protein at a pH of 6. Although these conditions favor peptide bond formation at the a-amino group (N-terminus) of the protein, the final product must be purified, as coupling of the peptide to non-terminal amino groups (lysine residues) of the protein may occur. The citraconyl groups may be removed from the conjugate by weak-acid hydrolysis (Dixon and Perham, 1968).

Backbone-engineered chemical ligation results in the conjugation of two peptides or proteins through the creation of a thioester or thioether linkage (Lindner and Robey, 1987; Robey and Fields, 1989). Thioester products are stable below a pH of 6, but are subject to hydrolysis at a pH greater than 7, rendering them inactive at physiological pH and therefore not useful in the scope of the present invention. Reacting a free thiol group on one peptide/protein with a bromoacetyl or iodoacetyl group on another will form a thioether linkage, which is stable at physiological and basic pH (Schnolzer and Kent, 1992). Specifically, the conjugation product of the present invention can be formed by ligating the bromoacetylated or iodoacetylated N-terminus of a protein to a modified RHAMM peptide. The RHAMM peptide is modified to contain a C-terminal Cysteine amide. When the modified peptide and protein are allowed to react, the thiol group on the peptide attacks the methylene carbon on the acetyl group of the protein, which releases the iodo (bromo) leaving group and results in a thioether linkage between the peptide and protein (Lloyd-Williams, 1997)

Heterobifunctional cross-linkers may be used to conjugate peptides/proteins where one molecule contains a free amine and the second molecule contains (or is modified to contain) a free thiol group and one such cross-linker is sulfo-GMBS (N-_-maleimidobutyryloxysulfo-succinimide ester) (Hermanson, 1996). A method for introducing a protected thiol into amine containing compounds uses a SAMA (S-acetylthioglycolic acid) group (Hermanson, 1996; Duncan, 1983). In the present method, the amine at the N-terminal of a protein is targeted to react with the sulfo-GMBS cross-linker through appropriate use of pH. The linkage between the protein and the cross-linker is a peptide bond and the remainder of the cross-linker contains a thiol reactive maleimide group. A RHAMM peptide is synthesized to contain the SAMA grouping at the N-terminal (through the use of a pentafluorophenol ester reactive intermediate during peptide synthesis) which can then be converted into a compound containing a free thiol in a reaction with hydroxylamine just prior to any conjugation experiments. The thus created RHAMM peptide (with reactive thiol) is combined with the modified protein (containing the thiol reactive maleimide group) to form the RHAMM-protein conjugate.”

Native chemical ligation involves the ligation of two unprotected peptides or proteins by formation of a thioester, followed by creation of an amide bond between the two sequences. For the purpose of the present invention, this involves ligation of the peptide C-terminus to the N-terminus of the protein. The process requires that the peptide has a C-terminal thioester group, and the protein must have an N-terminal cysteine residue. The reaction takes place at a pH of 6 in phosphate buffer with 6M quanidine. The free thiol group in the cysteine residue will attack the α-carbon of the thioester, resulting in an unstable intermediate that spontaneously rearranges to give an amide bond between the two sequences (Dawson, 1994; Lloyd-Williams et al., 1997).

A linkage sequence of amino acids may be added directly (during direct protein sequencing) or coded by insertion of adjacent nucleic acids (during recombinant synthesis) to the amino or carboxyl terminus of the peptide and protein. Alternatively, a non-peptide sequence may serve as the linkage between the peptide and protein. The purpose of the linkage molecule or sequence is to form a physical separation between the peptide and the protein in order to prevent steric hindrance between; increase flexibility of the bond between; facilitate attachment and detachment of; and prolong the half-life of; the peptide and protein. This technique is known to persons skilled in the art, and often yields more active complexes (Hermanson, 1996). Such linkage may be chosen from, but is not limited to, the group comprising: the constant domain of an antibody; the hinge region of an antibody; an amino acid sequence which permits enzymatic ligation of the two peptides (for example, when cysteine is required at the N-terminus of the protein for enzymatic ligation; polyethylene glycol; an amino acid sequence of sufficient length; and a chemical linkage and attached group.

Therapeutic Formulations of the Present Invention

The conjugate proteins of the invention may be formulated into pharmaceutical compositions for adminstration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, the route of drug administration and the ability of drug to elicit a desired response in the individual.

The active substance may be administered in a convenient manner such as injection (subcutaneous, intravenous, intramuscular, etc.), oral administration, inhalation, transdermal administration (such as topical cream or ointment, etc.), or suppository applications. Depending on the route of adminstration, the active substance may be coated in a material to protect the compound from the action of enzymes, acids and other natural conditions which may inactivate the compound. The nature of said coated preparations may range from conventional enteric-coated formulations (Wilding, I. R. et al., Pharmacol. Ther. 62: 97-124 (1994)) to liposomal or microsphere formulations consisting unilamellar or multilamellar vesicles containing the active substance for oral, parenteral or pulmonary administration. Prepararation of liposomes or microspheres have been described (Constantinides, P. P., Pharm. Res. 12: 1561-1572 (1995); Jones, M. N., Adv. Colloid Interface Sci. 54: 93-128 (1995); Cohen, S. et al., Int. J. Technol. Assess. Health Care 10: 121-130 (1994)); Kinnunen, P. K. et al., Chem. Phys. Lipids 73: 181-207 (1994). Extensive research to improve their physicochemical properties and pharmacokinetics have also been documented (Hnatyszyn, H. J. et al., PDA J. Pharm. Sci. Technol. 48: 247-254 (1994); Patel, N. M., Crit. Rev. Ther. Drug Carrier Syst. 9: 39-90 (1992)).

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The pharmaceutical compositions of the present invention may be used in methods for treating animals, including humans, with a disorder that can be treated by the corresponding therapeutic protein. Said compositions and preparations may contain a conjugate protein, either alone, or in combination with other active substances.

In particular, compositions for parenteral administration can be prepared as solutions of the conjugate protein or as powders of the conjugate protein to be mixed with one or more pharmaceutically acceptable excipients or diluents, suitable for the aforesaid uses and with an osmolarity which is compatible with the physiological fluids. For example, as described herein, said preparation may be formulated with a wetting agent and/or stabilized by addition of a stabilizer. When administering the compositions/preparations of the invention by injection, the administration may be by continuous infusion or by single or multiple injections. In an embodiment of the invention, forms for intravenous injection or infusion are selected to maximize drug availability, reduce dosage, and to elicit faster pharmacodynamic action.

The compositions and preparations of the invention are intended to provide to the recipient subjects an amount of a conjugated therapeutic protein sufficient to treat the symptoms of or prevent a disease or disorder treatable by the native therapeutic protein. Dosages of a conjugated protein to be administered depend largely on individual needs, on the desired effect in and duration a particular therapeutic indication, and on the chosen route of drug administration.

Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, the route of drug administration and the ability of drug to elicit a desired response in the individual. Determination of a a therapeutically active amount of the pharmaceutical compositions of the present invention also depends on the stoichiometric ratio for the number of HA-binding peptide or protein(s) conjugated to the number of therapeutic agent. In addition, the molecular size of the HA-binding protein will also affect dosage on a gram per body weight or body surface area basis.

For example, under the assumption that: (i) the HA-binding domain is a peptide with an amino acid sequence as set forth as SEQ ID NO.: 13; (ii) the therapeutic agent is granulocyte-macrophage colony stinulating factor (GM-CSF) or granulocyte colony stinulating factor (G-CSF); and (iii) the stoichiometric ratio between HA-binding peptide and therapeutic agent is 1:1, the recommended daily parenteral doses of the HA-bind-peptide-GM-CSF and HA-bind-peptide-G-CSF conjugates would range from about 0.01 μg/kg/day to about 30 μg/kg/day and preferably between 0.5 μg/kg/day to 10 μg/kg/day given by intravenous infusion or subcutaneous injection.

Similarly, if: (i) the HA-binding domain is a peptide with an amino acid sequence as set forth as SEQ ID NO.: 13; (ii) the therapeutic agent is erythropoietin; and (iii) the stoichiometric ratio between HA-binding peptide and therapeutic agent is 1:1, the recommended daily parenteral dose of HA-bind-peptide-erythropoietin conjugate would range from about 1 U/kg/day to 1000 U/kg/day and preferably between 3 U/kg/day to 300 U/kg/day given by intravenous or subcutaneous injection.

Further, if: (i) the HA-binding domain is a peptide with an amino acid sequence as set forth as SEQ ID NO.: 13; (ii) the therapeutic agent is human growth hormone; and (iii) the stoichiometric ratio between HA-binding peptide and therapeutic agent is 1:1, the recommended daily parenteral dose of HA-bind-peptide-human growth hormone conjugate would range from about 0.001 IU/kg/day to 10 IU/kg/day and preferably between 0.05 IU/kg/day to 0.2 IU/kg/day given by subcutaneous or intramuscular injection.

Yet further, if: (i) the HA-binding domain is a peptide with an amino acid sequence as set forth as SEQ ID NO.: 13; (ii) the therapeutic agent is an alpha-interferon; and (iii) the stoichiometric ratio between HA-binding peptide and therapeutic agent is 1:1, the recommended daily parenteral dose of HA-bind-peptide-alpha-interferon conjugate would range from about 0.001 MIU/kg/day to 1 MIU/kg/day and preferably between 0.005 IU/kg/day to 0.5 MIU/kg/day given by subcutaneous or intramuscular injection.

The term “antibody” as used herein is intended to include fragments thereof which also specifically react with a cell surface component. Antibodies can be fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab′ fragments.

Chimeric antibody derivatives, i.e. antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes a cell surface antigen (See, for example, Morrison et al., Proc. Natl Acad. Sci. U.S.A. 81:6851 (1985); Takeda et al., Nature 314:452 (1985), Cabilly et al., U.S. Pat. No. 4,816,567; Boss et al., U.S. Pat. No. 4,816,397; Tanaguchi et al., E.P. Patent No. 171,496; European Patent No. 173,494, United Kingdom Patent No. GB 2177096B). It is expected that chimeric antibodies would be less immunogenic in a human subject than the corresponding non-chimeric antibody.

Monoclonal or chimeric antibodies specifically reactive against cell surface components can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g. Teng et al., Proc. Natl. Acad. Sci. U.S.A., 80:7308-7312 (1983); Kozbor et al., Immunology Today 4:7279 (1983); Olsson et al., Meth. Enzymol., 92:3-16 (1982), and PCT Publication WO92/06193 or EP 239,400). Humanized antibodies can also be commercially produced (Scotgen Limited, 2 Holly Road, Twickenham, Middlesex, Great Britain.)

Specific antibodies, or antibody fragments, reactive against cell surface components may also be generated by screening expression libraries encoding immunoglobulin genes, or portions thereof; expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)). Alternatively, a SCID-hu mouse, for example the model developed by Genpharm, can be used to produce antibodies, or fragments thereof.

Dosage regime may be adjusted to provide the optimum therapeutic response. For example, the daily doses may be combined into a less frequent dosage regimen (e.g. to three-times weekly or t.i.w.), several divided doses may be administered on a given day (e.g. three-times per day or t.i.d.) or the dose may be proportionally reduced as indicated by the exigencies of the therapeutic situation.

Without further elaboration, it is believed that one skilled in the art can, using the preceding description, utilize the present invention to its fullest extent. The following specific embodiments are therefore to be construed as merely illustrative and not limiting of the remainder of the disclosure in any way whatsoever. The following examples serve as illustrations of the invention only, and are specifically directed to conjugation techniques and therapeutic proteins for use in the present invention.

### EXAMPLE 1

Production of RHAMM-EPO Direct Conjugate using Backbone Engineered Thioether Linkage

Erythropoietin would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated EPO to a RHAMM peptide will increase the half-life and enhance the tissue targeting of EPO and direct the conjugate to tissues with a high concentration of HA. It has been reported that approximately 25% of HA is found in bone, which is an optimal target for EPO, and as such, in addition to increasing half-life, conjugation to RHAMM may enhance target specificity and the therapeutic action of erythropoietin for example in the treatment of anemia.

Preparation of Recombinant EPO

The complete coding DNA sequence of human erythropoietin was first disclosed in U.S. patent application Ser. No. 655,841, filed Sep. 28, 1984, which was later issued as U.S. Pat. No. 4,703,008 [GenBank Accession No. AH009003 or E00630]. Recombinant EPO can be produced by methods known to persons skilled in the art, or purchased from existing commercial sources. For this method of coupling, the protein does not require modification or engineering prior to the conjugation step.

A sulfhydryl group must be added to the nitrogen of the lysine residue of EPO for attachment of the RHAMM peptide. This may be accomplished by dissolving EPO in 50 mM phosphate buffer and 150 mM NaCl at a pH of 8. 2-iminothiolane (2-IT) is added at a 10-fold molar excess, followed by incubation for one hour at room temperature (Hermanson, 1996; Traut et al., 1973). Following thiolation, the protein is purified by SEC and lyophilized. The preparation of recombinant EPO for thioether linkage conjugation to RHAMM is illustrated structurally below, where H2N— and —OH represent the N and C terminals of EPO, respectively.

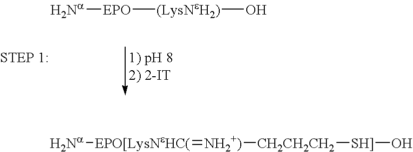

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention.

The RHAMM peptide must be engineered with an N-terminal bromoacetyl group, which will later be ligated to the thioloated lysine residues. To this end, the RHAMM peptide is coupled to a hydroxymethyl resin if a free acid is desired, or to a benzhydrylamine resin for a C-terminal amide. RHAMM is then synthesized as per normal Boc or Fmoc solid phase peptide synthesis (SPPS) methods. Following complete synthesis of the RHAMM peptide, the peptide is deprotected and coupled with bromoacetic anhydride (Robey, 1994; Robey and Fields, 1989). Finally, SEC and/or RP-HPLC methods are used to purify the peptide. The process is illustrated structurally below.

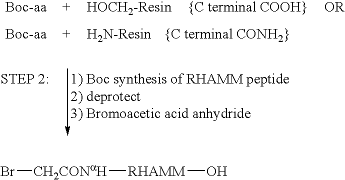

Coupling of the RHAMM Peptide and EPO

Thiolated EPO is dissolved in 0.1 M NaHCO₃. The solid bromoacetylated RHAMM peptide is added to the solution, the pH adjusted to 8, and the solution is allowed to incubate at room temperature for 3 hours (Robey and Fields, 1989, Kolodny and Robey, 1990, Muir et al., 1994). Purification of the conjugate is by SEC and/or RP-HPLC. The structural representation of the conjugation reaction is illustrated below.

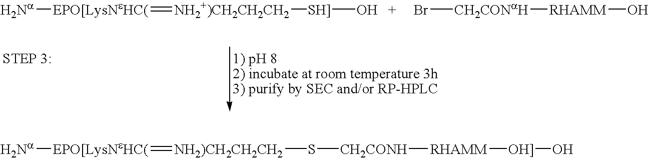

Characterization

The level of conjugation can be assessed by determining the mass of the conjugate, and a tryptic digestion map will determine the point of substitution.

### EXAMPLE 2

Conjugation of RHAMM Peptide and hGH by the Coupling of Minimally Protected Peptides

Human growth hormone (hGH) binds to cell-surface receptors throughout the body to stimulate organ and tissue growth, and of specific relevance to the present invention is the anabolic effect of hGH on bone formation in the epiphyses of long bones. hGH is therefore beneficial in growth insufficiencies and wasting disorders; however, excessive use of hGH has been associated with onset of diabetes and an increased incidence of osteoporosis and heart disease. The conjugation of hGH to RHAMM peptide is expected to increase distribution of hGH to the bone marrow, allowing a reduction in dosage requirements, and a decreased incidence of insulin-like side effects with respect to glucose metabolism as well as other hGH-induced toxicities.

Preparation of Recombinant hGH

The complete coding sequence of human growth hormone is given as GenBank Accession No. E00141, E01424 or A15072. For this method of coupling, the protein does not require modification or engineering prior to the conjugation step. Recombinant hGH may be purchased from existing commercial sources, or produced by methods known to persons skilled in the art.

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention.

The RHAMM peptide and is synthesized with a C-terminal thiocarboxyl moiety, which may be accomplished by standard SPPS methods using the following procedure. Boc-thioglycine is coupled to a carboxyl handle, and then to an aminomethyl resin in the presence of dicyclohexylcarbodiimide (DCC) (Baca, 1995, Blake, 1981, and Yamashiro, 1988). The RHAMM peptide can then be synthesized using standard Boc SPPS protocols and cleaved from the resin with HF to give RHAMM-Gly-thiocarboxyl. Prior to conjugation of the engineered RHAMM peptide with hGH, the amino groups of RHAMM must be blocked. This is accomplished by dissolving the modified RHAMM peptide in potassium phosphate buffer at pH 7.5, followed by the addition of citraconic anhydride dissolved in dioxane. The modified and blocked RHAMM peptide is then purified by SEC in 50 mM ammonium bicarbonate and lyophilized. The preparation of the RHAMM peptide for silver ion activation of the thiocarboxyl C-terminus is illustrated structurally below.

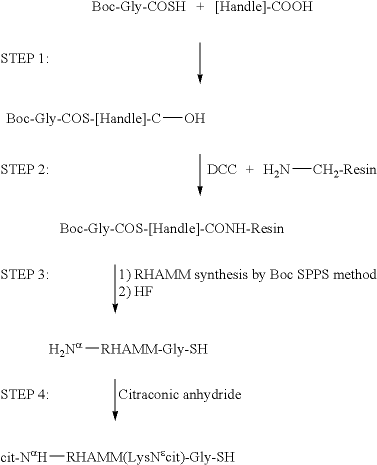

Coupling of RHAMM Peptide and hGH

For the coupling of the RHAMM peptide to the ε amino groups on the lysine residues of the protein, conjugation should take place at a pH of 8-9. If conjugation between the RHAMM peptide and predominantly the α amino group (N-terminus) of the protein is preferred, conjugation should take place at pH 6-7.

Both peptide and protein are dissolved in water and pH adjusted as desired. This is followed by the addition of N-hydroxysuccinimide at 0° C. (also at desired pH). AgNO₃ is added, and the solution is incubated at 0° C. for 30 minutes and at room temperature for 45 minutes (Blake and Li, 1981, 1983). The protective citraconyl groups are removed by incubation with a large excess of 6M acetic acid for 2.5 hours. The final product is lyophilized and purified by RP-HPLC using water/TFA and acetonitrile/TFA. The product is eluted by buffer exchange into an appropriate buffer using SEC. The coupling reaction between hGH and prepared RHAMM peptide is illustrated structurally below.

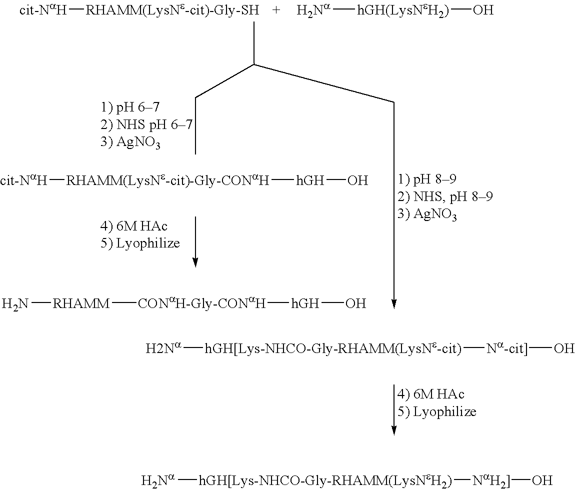

Characterization

The level of conjugation can be assessed by determining the mass of the conjugate, and a tryptic digestion map will determine the point of substitution (if not N-terminal).

### EXAMPLE 3

Conjugation of RHAMM Peptide to hGH by Backbone Engineered Thioether Linkage using a Bifunctional PEG Linkage

Preparation of Recombinant hGH

The complete coding sequence of human growth hormone is given as GenBank Accession No. E00141, E01424 or A15072. For this method of coupling, the protein does not require modification or engineering prior to the conjugation step. Recombinant hGH may be purchased from existing commercial sources, or produced by methods known to persons skilled in the art.

A sulfhydryl group must be added to the e nitrogen of the lysine residue of hGH for attachment of the RHAMM peptide. This may be accomplished by dissolving hGH in 50 mM phosphate buffer and 150 mM NaCl at a pH of 8. 2-iminothiolane (2-IT) is added at a 10-fold molar excess, followed by incubation for one hour at room temperature (Hermanson, 1996; Traut et al., 1973). Following thiolation, the protein is purified by SEC and lyophilized. The preparation of recombinant hGH for thioether linkage conjugation to RHAMM is illustrated structurally below.

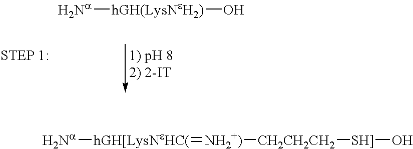

Preparation of N-Terminal Bromoacetylated RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide-PEG conjugate must be engineered with an N-terminal bromoacetyl group, which will later be ligated to the lysine residues on hGH that have been thiolated. To this end, the RHAMM peptide is coupled to a hydroxymethyl resin if a free acid is desired, or to a benzhydrylamine resin for a C-terminal amide. RHAMM is then synthesized as per normal Fmoc SPPS methods (Boc methods are not recommended for this procedure (Lu and Felix, 1993)). Following complete synthesis of the RHAMM peptide, the bifunctional PEG molecule is coupled to the fully synthesized RHAMM peptide as per standard Fmoc SPPS methods. The N-terminal of the RHAMM-PEG conjugate is then deprotected and coupled with bromoacetic anhydride. Finally, SEC and/or RP-HPLC methods are used to purify the peptide. The process is illustrated structurally below.

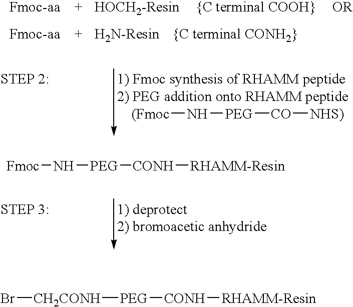

Coupling of RHAMM-PEG to hGH

Tiolated hGH is dissolved in 0.1 M NaHCO₃. The bromoacetylated RHAMM-PEG conjugate is added to the solution, the pH adjusted to 8, and the solution is allowed to incubate at room temperature for 3 hours (Robey and Fields, 1989, Kolodny and Robey, 1990, Muir et al., 1994). Purification of the conjugate is by SEC and/or RP-HPLC. The structural representation of the conjugation reaction is illustrated below.

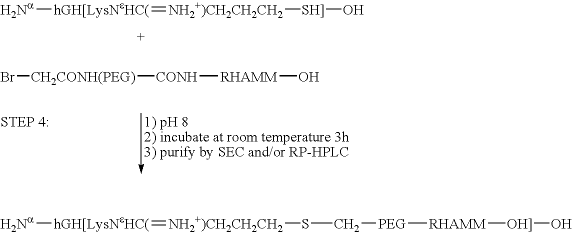

A dosage of 0.06-0.1 mg (0.16-0.026 IU) hGH per kg body weight 3 times weekly is recommended for the treatment of growth deficiency associated with growth hormone insufficiency. For the treatment of growth failure in patients with Turner's syndrome, dosage of up to 1 IU/kg/week is recommended (or 24 IU/m2). Adverse effects of hGH treatment include antibody production, intracranial hypertension, and increased incidences of tumor and leukemia. Excessive use of hGH has been associated with onset of diabetes and an increased incidence of osteoporosis and heart disease.

### EXAMPLE 4

Conjugation of RHAMM Peptide to EPO by Coupling of Minimally Protected Peptides by Silver ion Activation of Thiocarboxyl C-Terminus using a Bifunctional PEG Molecule as a Linker

Preparation of Recombinant EPO

The complete coding DNA sequence of human erythropoietin was first disclosed in U.S. patent application Ser. No. 655,841, filed Sep. 28, 1984, which was later issued as U.S. Pat. No. 4,703,008 [GenBank Accession No. AH009003 or E00630]. Recombinant EPO can be produced by methods known to persons skilled in the art, or purchased from existing commercial sources. For this method of coupling, the protein does not require modification or engineering prior to the conjugation step.

Recommended dose of EPO ranges from 75 U/kg to 600 U/kg thrice weekly depending on the indication. Adverse effects include allergic reaction and antibody induction, clotting of dialyzer/artificial kidney, hypertension, seizures, headache, arthralgia, tachycardia, nausea, clotted vascular access, shortness of breath, hyperkalemia, and diarrhea.

Preparation of RHAMM-PEG

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide and is synthesized with a C-terminal thiocarboxyl moiety, which may be accomplished by incorporating a bifunctional PEG molecule onto the end of RHAMM. The PEG molecule serves both as a thiocarboxyl terminal group, and as a physically extended linkage between RHAMM and EPO. Fmoc-thioglycine is coupled to a carboxyl handle, and then to an aminomethyl resin in the presence of dicyclohexylcarbodiimide (DCC) (Baca, 1995, Blake, 1981, and Yamashiro, 1988). The bifunctional PEG molecule is then coupled to RHAMM as per normal Fmoc SPPS. The PEG-RHAMM molecule is cleaved from the resin with HF to give RHAMM-PEG-Gly-SH. Prior to conjugation of the engineered RHAMM-PEG with EPO, the amino groups of RHAMM must be blocked. This is accomplished by dissolving the RHAMM-PEG conjugate in potassium phosphate buffer at pH 7.5, followed by the addition of citraconic anhydride dissolved in dioxane. The modified and blocked RHAMM-PEG conjugate is then purified by SEC in 50 mM ammonium bicarbonate and lyophilized. The preparation of the RHAMM peptide for silver ion activation of the thiocarboxyl C-terminus is illustrated structurally below.

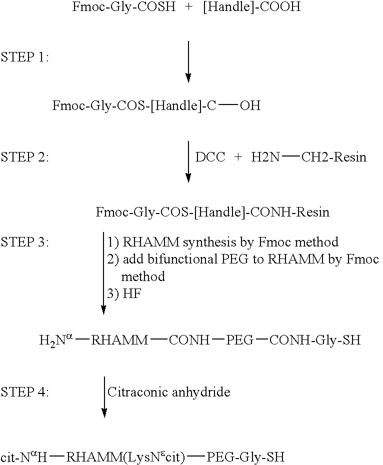

Conjugation of RHAMM-PEG to EPO

For the coupling of the RHAMM-PEG to the ε amino groups on the lysine residues of the protein, conjugation should take place at a pH of 8-9. If conjugation between RHAMM-PEG and predominantly the α amino group (N-terminus) of the protein is preferred, conjugation should take place at pH 67.

Both peptide and EPO are dissolved in water and pH adjusted as desired. This is followed by the addition of N-hydroxysuccinimide at 0° C. (also at desired pH). AgNO₃ is added, and the solution is incubated at 0° C. for 30 minutes and at room temperature for 45 minutes (Blake and Li, 1981, 1983). The protective citraconyl groups are removed by incubation with a large excess of 6M acetic acid for 2.5 hours. The final product is lyophilized and purified by RP-HPLC using water/TFA and acetonitrile/TFA. The product is eluted by buffer exchange into an appropriate buffer using SEC. The coupling reaction between EPO and prepared RHAMM-PEG is illustrated structurally below.

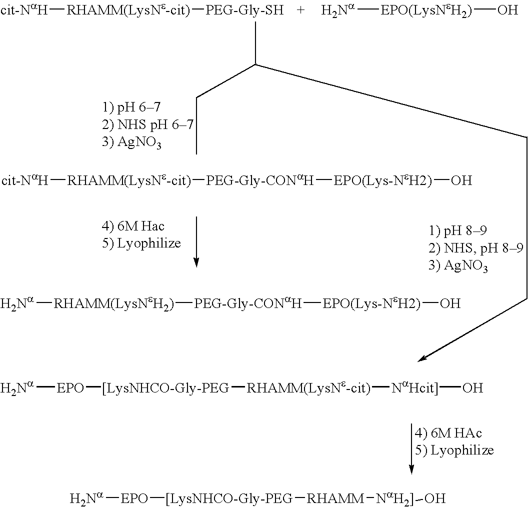

### EXAMPLE 5

Conjugation of RHAMM Peptide and GM-CSF by the Coupling of Minimally Protected Peptides

The colony stimulating factors (G-CSF and GM-CSF) are natural haematopoietic growth factors that regulate the production and functional activity of granulocytes, macrophages, and mature white cells. These white cells form the basis of the body's immune system, which may be compromised by events ranging from chemotherapy or infection to idiopathic neutropenia. These agents are useful in reducing the duration of hospitalization, intensity of hospitalized treatment, and antibiotic usage among cancer patients, improve overall prognosis, and permit higher, more frequent doses of chemotherapy. Therefore, G-CSF and GM-CSF are useful in the treatment of cancer, and may be used to stimulate proliferation of cell colonies in various tissues. The conjugation of a CSF to RHAMM would exploit the distribution of endogenous HA, allowing RHAMM to direct the CSF to tissues rich in HA, including the lymph nodes and bone marrow. This technology would be useful in the mobilization of HIV, which lies dormant in lymph nodes following antiretroviral treatment. The specific targeting of the CSF to the lymph nodes is expected to stimulate the proliferation of cells harboring the latent virus, allowing the antiretroviral therapy to eradicate the virus. As the majority of hematopoiesis occurs in bone marrow, distribution of the CSF to the bone marrow would stimulate myeloid reconstitution and stem cell mobilization following destruction of immune cells, such as in chemotherapy, neutropenia, and bone marrow or stem cell transplantation.

Another application of a CSF-RHAMM conjugate would be in cancer metastasis, when tumor cells migrate to the lymph nodes. Increased concentrations of HA have been documented in cancer cells as well as in lymph nodes towards which a conjugate of the present invention could target. Such a conjugate would preferably contain a HA-binding domain linked directly or indirectly to tumor necrosis factor (TNF), interleukin (IL), interferon (IFN), and/or other chemotherapeutic agents.

The recommended daily parenteral doses of GM-CSF and G-CSF range from about 1 μg/kg/day to about 20 μg/kg/day preferably for 21 days. The injection is generally administered in 2-hour i.v. infusions, or as subcutaneous injections. At these doses, notable low-grade toxicities include headache, peripheral edema, arthralgia, myalgia, and skin rash. Pericarditis is the major dose-limiting side effect for GM-CSF at high dosages, and is contraindicated in patients with excessive myeloblasts in bone marrow or known hypersensitivity to expression-host proteins.

The recommended dose of G-CSF is 5-10 μg/kg/day for up to 21 days. Major low-grade adverse reactions associated with G-CSF therapy include medullary bone pain, and spontaneously reversible elevations in uric acid, lactate dehydrogenase, and alkaline phosphatase. The only reported dose-limiting side effect of G-CSF therapy is Sweet syndrome (acute febrile neutrophilic dermatosis).

Preparation of Recombinant GM-CSF

The complete coding sequence of human GM-CSF is given as GenBank Accession No. E00951. For this method of coupling, the protein does not require modification or engineering prior to the conjugation step. Recombinant GM-CSF may be purchased from existing commercial sources, or produced by methods known to persons skilled in the art.

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide and is synthesized with a C-terminal thiocarboxyl moiety, which may be accomplished by standard SPPS methods using the following procedure. Boc-thioglycine is coupled to a carboxyl handle, and then to an aminomethyl resin in the presence of dicyclohexylcarbodiimide (DCC) (Baca, 1995, Blake, 1981, and Yamashiro, 1988). The RHAMM peptide can then be synthesized using standard Boc SPPS protocols and cleaved from the resin with HF to give RHAMM-Gly-thiocarboxyl. Prior to conjugation of the engineered RHAMM peptide with hGH, the amino groups of RHAMM must be blocked. This is accomplished by dissolving the modified RHAMM peptide in potassium phosphate buffer at pH 7.5, followed by the addition of citraconic anhydride dissolved in dioxane. The modified and blocked RHAMM peptide is then purified by SEC in 50 mM ammonium bicarbonate and lyophilized. The preparation of the RHAMM peptide for silver ion activation of the thiocarboxyl C-terminus is illustrated structurally below.

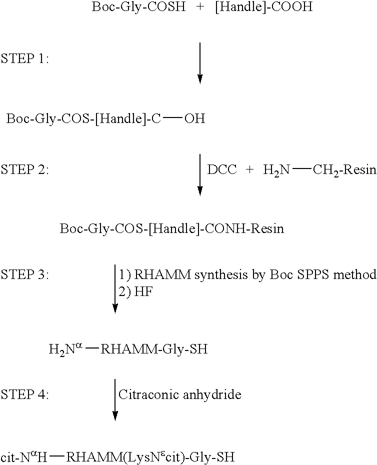

Coupling of RHAMM Peptide and hGM-CSF

For the coupling of the RHAMM peptide to the ε amino groups on the lysine residues of the protein, conjugation should take place at a pH of 8-9. If conjugation between the RHAMM peptide and predominantly the α amino group (N-terminus) of the protein is preferred, conjugation should take place at pH 6-7.

Both peptide and protein are dissolved in water and pH adjusted as desired. This is followed by the addition of N-hydroxysuccinimide at 0° C. (also at desired pH). AgNO₃ is added, and the solution is incubated at 0° C. for 30 minutes and at room temperature for 45 minutes (Blake and Li, 1981, 1983). The protective citraconyl groups are removed by incubation with a large excess of 6M acetic acid for 2.5 hours. The final product is lyophilized and purified by RP-HPLC using water/TFA and acetonitrile/TFA. The product is eluted by buffer exchange into an appropriate buffer using SEC. The coupling reaction between hGM-CSF and prepared RHAMM peptide is illustrated structurally below.

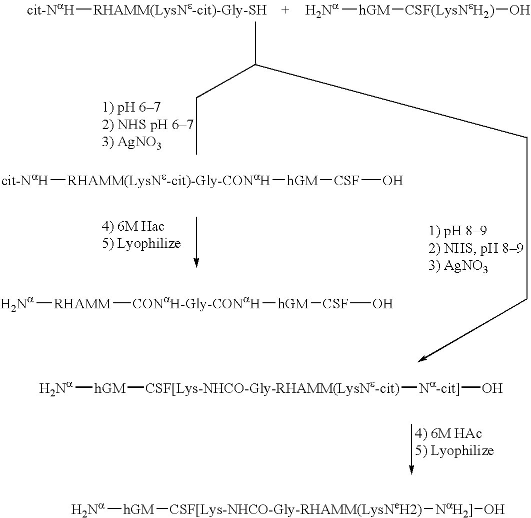

Characterization

The level of conjugation can be assessed by determining the mass of the conjugate, and a tryptic digestion map will determine the point of substitution (if not N-terminal).

### EXAMPLE 6

Conjugation of RHAMM Peptide and Parathyroid Hormone (PTH) by the Coupling of Minimally Protected Peptides

Parathyroid hormone has been used therapeutically to increase bone mineral density in osteoporosis, corticosteroid-induced osteoporosis, estrogen deficiency-related bone loss, and hypoparathyroidism. The conjugation of a PTH to RHAMM would exploit the distribution of endogenous HA, allowing RHAMM to direct PTH to its main target of therapeutic action, the bones. Administration of PTH has been associated with various nonspecific side effects, which may not be produced following administration of a PTH-RHAMM conjugate.

Preparation of Recombinant PTH

The complete coding sequence of human PTH is given as GenBank Accession No. E01147, A08523, A08525 or A08533. For this method of coupling, the protein does not require modification or engineering prior to the conjugation step. Recombinant PTH may be purchased from existing commercial sources, or produced by methods known to persons skilled in the art.

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide and is synthesized with a C-terminal thiocarboxyl moiety, which may be accomplished by standard SPPS methods using the following procedure. Boc-thioglycine is coupled to a carboxyl handle, and then to an aminomethyl resin in the presence of dicyclohexylcarbodiimide (DCC) (Baca, 1995, Blake, 1981, and Yamashiro, 1988). The RHAMM peptide can then be synthesized using standard Boc SPPS protocols and cleaved from the resin with HF to give RHAMM-Gly-thiocarboxyl. Prior to conjugation of the engineered RHAMM peptide with hGH, the amino groups of RHAMM must be blocked. This is accomplished by dissolving the modified RHAMM peptide in potassium phosphate buffer at pH 7.5, followed by the addition of citraconic anhydride dissolved in dioxane. The modified and blocked RHAMM peptide is then purified by SEC in 50 mM ammonium bicarbonate and lyophilized. The preparation of the RHAMM peptide for silver ion activation of the thiocarboxyl C-terminus is illustrated structurally below.

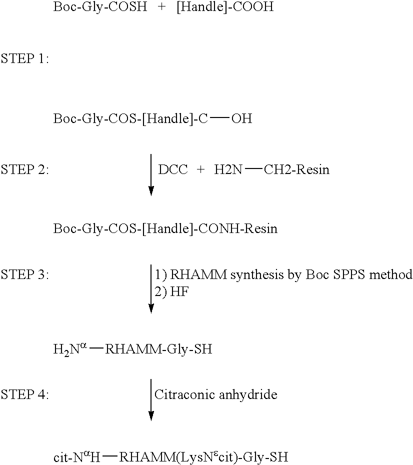

Coupling of RHAMM Peptide and hPTH

For the coupling of the RHAMM peptide to the ε amino groups on the lysine residues of the protein, conjugation should take place at a pH of 8-9. If conjugation between the RHAMM peptide and predominantly the α amino group (N-terminus) of the protein is preferred, conjugation should take place at pH 6-7.

Both peptide and protein are dissolved in water and pH adjusted as desired. This is followed by the addition of N-hydroxysuccinimide at 0° C. (also at desired pH). AgNO₃ is added, and the solution is incubated at 0° C. for 30 minutes and at room temperature for 45 minutes (Blake and Li, 1981, 1983). The protective citraconyl groups are removed by incubation with a large excess of 6M acetic acid for 2.5 hours. The final product is lyophilized and purified by RP-HPLC using water/TFA and acetonitrile/TFA. The product is eluted by buffer exchange into an appropriate buffer using SEC. The coupling reaction between hPTH and prepared RHAMM peptide is illustrated structurally below.

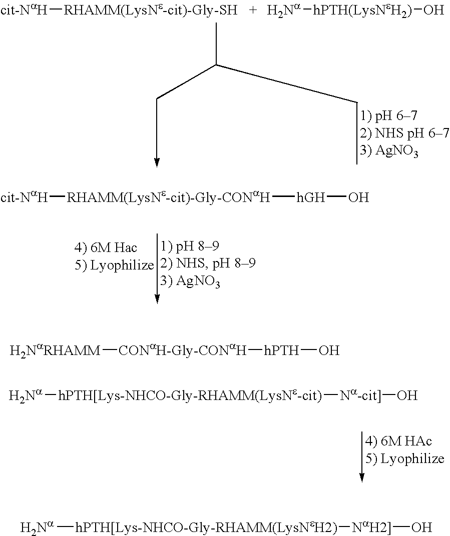

Characterization

The level of conjugation can be assessed by determining the mass of the conjugate, and a tryptic digestion map will determine the point of substitution (if not N-terminal).

### EXAMPLE 7

Production of RHAMM-Amantadine Direct Conjugate using Thioether Linkage

Preparation of Amantadine

Amantadine was developed in the 1960s and has diverse therapeutic utiltities ranging from prevention of influenza A infection to the treatment of Parkinson's disease (Aoki and Sitar, 1988). 1-Aminoadamantane hydrochloride (amantadine hydrochloride) is available commercially as an antiviral under the name Symmetrel (E. I. du Pont de Nemours and Company, Wilmington, Del.). Amantadine hydrochloride may also be prepared as known in the art, as described in U.S. Pat. No. 3,310,469.

A sulfhydryl group must be added to the nitrogen of the primary amine group of amantadine for attachment of the RHAMM peptide. This may be accomplished by dissolving amantadine in 50 mM phosphate buffer and 150 mM NaCl at a pH of 8. 2-iminothiolane (2-IT) is added at a 10-fold molar excess, followed by incubation for one hour at room temperature (Hermanson, 1996; Traut et al., 1973). Following thiolation, the thiolated amantadine is purified by SEC and lyophilized. The preparation of amantadine for thioether linkage conjugation to RHAMM is illustrated structurally below.

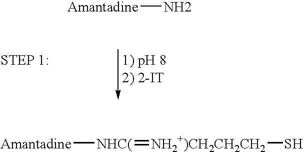

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide must be engineered with an N-terminal bromoacetyl group, which will later be ligated to the thioloated primary amine. To this end, the RHAMM peptide is coupled to a hydroxymethyl resin if a free acid is desired, or to a benzhydrylamine resin for a C-terminal amide. RHAMM is then synthesized as per normal Boc or Fmoc solid phase peptide synthesis (SPPS) methods. Following complete synthesis of the RHAMM peptide, the peptide is deprotected and coupled with bromoacetic anhydride (Robey, 1994; Robey and Fields, 1989). Finally, SEC and/or RP-HPLC methods are used to purify the peptide. The process is illustrated structurally below.

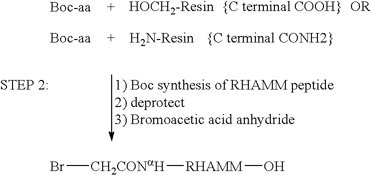

Coupling of the RHAMM Peptide and Amantadine

Thiolated amantadine is dissolved in 0.1 M NaHCO₃. If solubility in aqueous solution is problematic, this could be circumvented by firt dissolving the small molecule drug in a small percentage of organic acid. The solid bromoacetylated RHAMM peptide is added to the solution, the pH adjusted to 8, and the solution is allowed to incubate at room temperature for 3 hours (Robey and Fields, 1989, Kolodny and Robey, 1990, Muir et al., 1994). Purification of the conjugate is by SEC and/or RP-HPLC. The structural representation of the conjugation reaction is illustrated below.

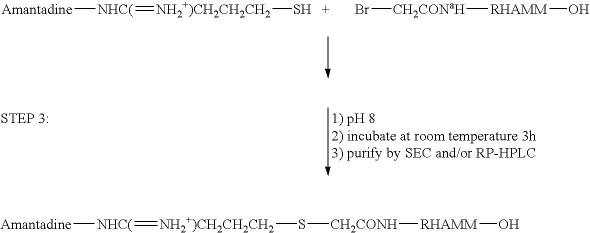

Characterization

The level of conjugation can be assessed by determining the mass of the conjugate, and a tryptic digestion map will determine the point of substitution.

### EXAMPLE 8

Conjugation of HA-Binding Domain to Antiviral Agents using Backbone Engineered Thioether Linkage

Preparation of Acyclovir and Ganciclovir

Acyclovir (acycloguanosine, 2-Amino-1,9-dihydro-9-[(2-hydroxyethoxy)methyl]-6H-purin-6-one) and ganciclovir (DHPG) are synthetic acyclic purine nucleoside analogs with antiviral activity. Acycloviris sold under the tradename Zovirax™ (Glaxo Wellcome) for the treatment of herpes simplex, chicken pox and zoster infections while Ganciclovir is sold under the tradename Cytovene™ (Roche) for the treatment of cytomegalovirus infections. Both products are available commercially and may also be sourced from Sigma (Sigma product numbers A250 and G2536 respectively). Acyclovir and ganciclovir may also be prepared as known in the art, as described in U.S. Pat. Nos. 4,544,634 and 5,994,321.

For the present example, a sulfhydryl group must be added to the nitrogen of the amino group of acyclovir or gangiclovir for attachment of the RHAMM peptide. This may be accomplished by dissolving acyclovir or gangiclovir in 50 mM phosphate buffer and 150 mM NaCl at a pH of 8. If solubility in aqueous solution is problematic, this could be circumvented by firt dissolving the small molecule drug in a small percentage of organic acid. 2-iminothiolane (2-IT) is added at a 10-fold molar excess, followed by incubation for one hour at room temperature (Hermanson, 1996; Traut et al., 1973). Following thiolation, the thiolated product is purified by SEC and lyophilized. The preparation of acyclovir or gangiclovir for thioether linkage conjugation to RHAMM is illustrated structurally below.

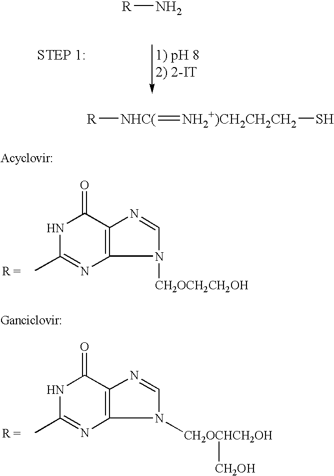

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. The RHAMM peptide must be engineered with an N-terminal bromoacetyl group, which will later be ligated to the thioloated lysine residues. To this end, the RHAMM peptide is coupled to a hydroxymethyl resin if a free acid is desired, or to a benzhydrylamine resin for a C-terminal amide. RHAMM is then synthesized as per normal Boc or Fmoc solid phase peptide synthesis (SPPS) methods. Following complete synthesis of the RHAMM peptide, the peptide is deprotected and coupled with bromoacetic anhydride (Robey, 1994; Robey and Fields, 1989). Finally, SEC and/or RP-HPLC methods are used to purify the peptide. The process is illustrated structurally below.

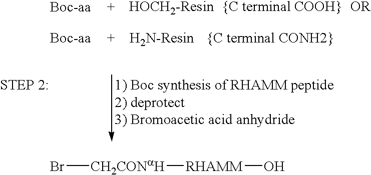

Coupling of the RHAMM Peptide and Acyclovir or Gangiclovir

Thiolated acyclovir or gangiclovir is dissolved in 0.1 M NaHCO₃. The solid bromoacetylated RHAMM peptide is added to the solution, the pH adjusted to 8, and the solution is allowed to incubate at room temperature for 3 hours (Robey and Fields, 1989, Kolodny and Robey, 1990, Muir et al., 1994). Purification of the conjugate is by SEC and/or RP-HPLC. The structural representation of the conjugation reaction is illustrated below.

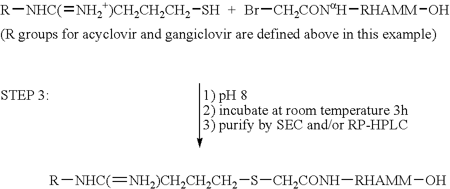

Characterization

The level of conjugation can be assessed by determining the mass of the conjugate, and a tryptic digestion map will determine the point of substitution.

### EXAMPLE 9

Conjugation of HA-Binding Domain to Anti-Cancer Agent using Direct Thioether Linkage

Preparation of Methotrexate and Cytarabine

Methotrexate, N-[4-[[(2,4-diamino-6-p teridinyl) methyl]methylamino]benzoyl]-L-glutamic acid, is one of the oldest chemotherapy drugs which is used alone or in combination with other chemotherapies, surgery or radiation for the treatment of cancers, lymphomas, leukemias, rheumatoid arthritis, and severe psoriasis. Cytarabine, 4-amino-1-β-D-arabinofuranosyl-2 (1H)-pyrimidinone or Cytosine arabinoside, is indicated for remission induction in acute non-lymphocytic leukemia of adults and pediatric patients. It has also been found useful in the treatment of acute lymphocytic leukemia and the blast phase of chronic myelocytic leukemia. Intrathecal administration of CYTOSAR-U is indicated in the prophylaxis and treatment of meningeal leukemia. Both products are available commercially and may also be sourced from Sigma (Sigma product numbers M9929 and C1768 respectively). Methotrexate and cytarabine may also be prepared as known in the art, as described in U.S. Pat. Nos. 4,080,325, 4,224,446, 4,374,987 and 5,641,758.

A sulfhydryl group must be added to the nitrogen of the amino group of methotrexate or cytarabine for attachment of the RHAMM peptide. This may be accomplished by dissolving methotrexate or cytarabine in. 50 mM phosphate buffer and 150 mM NaCl at a pH of 8. If solubility in aqueous solution is problematic, this could be circumvented by firt dissolving the small molecule drug in a small percentage of organic acid. 2-iminothiolane (2-IT) is added at a 10-fold molar excess, followed by incubation for one hour at room temperature (Hermanson, 1996; Traut et al., 1973). Following thiolation, the thiolated product is purified by SEC and lyophilized. The preparation of methotrexate or cytarabine for thioether linkage conjugation to RHAMM is illustrated structurally below.

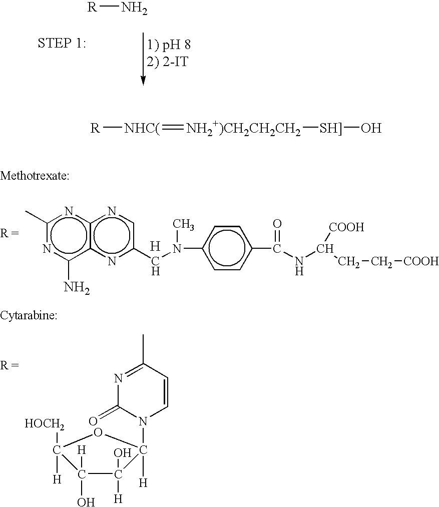

Preparation of RHAMM Peptide

For the purpose of illustration in this example, the HA-binding domain has an amino acid sequence as defined in SEQ. I.D. 13. It should be appreciated by those skilled in the art that other amino acid sequences representing other HA-binding peptides or polypeptides as contemplated hereunder shall constitute equivalent substitutions without departure from the principles of the present invention. Furthermore, the following method is a representation of a multitude of methods as known in the art for conjugation of a polypeptide to an anti-cancer agent, for example, as described in U.S. Pat. No. 5,108,987.

In this instance, the RHAMM peptide must be engineered with an N-terminal bromoacetyl group, which will later be ligated to the thioloated lysine residues. To this end, the RHAMM peptide is coupled to a hydroxymethyl resin if a free acid is desired, or to a benzhydrylamine resin for a C-terminal amide. RHAMM is then synthesized as per normal Boc or Fmoc solid phase peptide synthesis (SPPS) methods. Following complete synthesis of the RHAMM peptide, the peptide is deprotected and coupled with bromoacetic anhydride (Robey, 1994; Robey and Fields, 1989). Finally, SEC and/or RP-HPLC methods are used to purify the peptide. The process is illustrated structurally below.

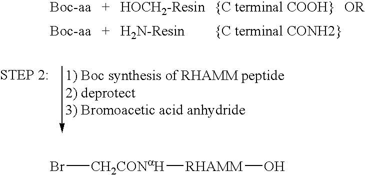

Coupling of the RHAMM Peptide and Methotrexate or Cytarabine

Thiolated methotrexate or cytarabine is dissolved in 0.1 M NaHCO3. The solid bromoacetylated RHAMM peptide is added to the solution, the pH adjusted to 8, and the solution is allowed to incubate at room temperature for 3 hours (Robey and Fields, 1989, Kolodny and Robey, 1990, Muir et al., 1994). Purification of the conjugate is by SEC and/or RP-HPLC. The structural representation of the conjugation reaction is illustrated below.

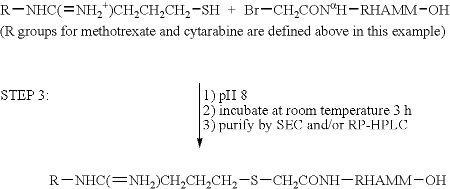

Characterization

The level of conjugation can be assessed by determining the mass of the conjugate, and a tryptic digestion map will determine the point of substitution.

### EXAMPLE 10

The in vitro HA-binding potency of any RHAMM conjugate can be assessed using an activated sepharose column. The sepharose is prepared according to the manufacturer's instructions and mixed with HA, resulting in a medium that can be used as an affinity column. The radiolabelled RHAMM conjugate is passed through the column, and the column is washed. If the HA-binding site of the RHAMM peptide conjugate remains accessible to HA, the conjugate will remain bound to the sepharose and will not be present in the eluent. The fraction of RHAMM peptide that remains bound to the column can be calculated based on the radioactivity of the column and eluent following washing of the column with buffer. This result is then compared with the results of passing unconjugated radiolabelled RHAMM peptide through an identical column, to determine the HA-binding ability of the conjugate.

### EXAMPLE 11

In addition to the previous experiment, the potency of the RHAMM conjugate can be further determined by a competitive binding experiment. The column is prepared by mixing the activated sepharose with hyluronan, and RHAMM peptide is then added to the column along with the radiolabelled RHAMM conjugate. In this manner, the potency of the conjugate can be compared with that of RHAMM peptide alone.

The pharmacokinetics and distribution of the RHAMM-protein conjugates can be determined in rabbits, rats, mice, or other animals by various methods known to persons skilled in the art. One experimental design may involve the parenteral injection of the conjugate molecule into rabbits and mice at predetermined doses. Twelve rabbits are to be included in the preliminary intravenous study to evaluate in vivo profiles of the conjugate molecules, with 6 rabbits in each group. The concentrations of radiolabelled test peptides in plasma will be determined at 5, 15, 30, and 45 minutes, and at 1, 2, 3, 6, 12, and 24 hours following intravenous administration by radioactivity counting.

The single dose study will be carried out in 2 groups of mice, one group allocated to intravenous injection, and one group to intramuscular injection. Sampling times will be determined based on results from the preliminary study in rabbits. Five test animals will be sacrificed at each sampling time and blood and organs will be collected. Urine and feces will also be collected in metabolic cages. The concentrations of the conjugate in various tissues will be analyzed by radioactivity counting.

A peptide having an identical amino acid composition to the conjugate will be administered under the same protocol to a third group of mice. The results from this experimental group will be compared to that of the animals receiving the conjugate molecule.

All animals will be euthanized after the study, and various organs (liver, kidney, heart, spleen, brain, lung, and bones) collected for drug distribution analysis. Basic pharmacokinetic parameters including elimination half-life, clearance, and volume of distribution will be calculated following intravenous injection using standard equations and methods known to those persons in the field of pharmacokinetics. The parameters determined following intramuscular injection shall include elimination half-life, clearance, volume of distribution, peak concentration and time, and bioavailability.

### EXAMPLE 12

The biological activity of GMCSF or a GMCSF-RHAMM conjugate can be determined by an in vitro bioassay using the TF-1 cell line and the MTT cell proliferation method. TF-1 cells are used in this assay, as it is a cell line of immature erythroid origin that proliferates dependently on GM-CSF.

One day prior to bioassay, the number of TF-1 cells necessary for growth factor deprivation must be calculated (4×10⁴ cells are needed/assay well). Cells are centrifuged at 270×g for 5 minutes and resuspended in culture media. This process is repeated, the supernatant aspirated, and the cells are resuspended in culture media to a concentration of 2×10⁵ cells/mL. The cell suspension is incubated at 37° C,, 8% CO₂ for 20-24 hours. Culture media can be prepared by dissolving 50 ml heat-inactivated fetal bovine serum (HI-FBS) in 500 ml DMEM (Gibco catalogue #11965-050), with 10 μl 2.5M 2-mercaptoethanol.

On day zero of the bioassay, the viable cell count of the growth factor-deprived cells is determined, and the volume of cells necessary for the assay is obtained (4×10⁴ cells/assay well). The cells are centrifuged at 270×g for 5 minutes, followed by aspiration of the supernatant and resuspension of the cell pellet in 2 volumes of fresh assay medium. Cells are again centrifuged, the supernatant is aspirated, and the pellet is resuspended in assay medium to a concentration of 4×10⁵ cell/mL.

For preparation of the standard curve, the GMCSF standard is diluted with assay medium to a concentration of 600 IU/mL, and serial dilutions are performed for determination of the standard curve. Assay medium is prepared in RPMI without phenol red (Gibco catalogue #11835-030), to a final concentration of 1 mg/ml insulin, 1 mg/ml transferrin, and 1×10⁴M 2-mercaptoethanol. Insulin and transferrin stock solutions are diluted first in Dulbecco's phosphate buffered saline (D-PBS) to 25 mg/ml, and 2-mercaptoethanol is mixed with gentamicin to 0.05M. Each test sample is prepared and diluted appropriately with sterilized PW to obtain a protein concentration of 0.4-0.6 mg/ml. The protein concentration is then calculated, and the process is repeated as required to obtain a dilution which gives a protein concentration of 0.4-0.6 mg/mL. This sample is then diluted 50× with 0.1% BSA in D-PBS. This will result in a GM-CSF concentration of approximately 10 ng/mL.

Eight 2-fold serial dilutions of the GM-CSF samples in assay medium are performed for determination of the dose-response curve. For the assay, approximately 4×10⁴ cells are added to each well with 50 ml of sample, standard, or blank. The plates are incubated at 37° C., 5% CO₂ for 72 hours. After incubation, MTT solution (4.0 mg/ml in D-PBS) is added to a concentration of 2 mg/ml, and the plate is sealed and mixed for 30 seconds, followed by incubation for 3 hours at 37° C., 5% CO₂. After incubation, the plate is centrifuged at 1775×g for 15 minutes. The supernatant is aspirated from all plates and 50 ml isopropanol is added to each well, followed by mixing of the sealed plates for 5 minutes. Optical density is read at 595 nm. The final potency of the sample is then calculated as follows:

$$Potency of test sample ( IU \text{/} mg ) = \frac{IU giving 50 \% maximal response ( 7.9 )}{mg of test sample giving 50 \% max . response ( 7.15 )}$$

### EXAMPLE 13

The biological activity of EPO samples can be determined by an in-vitro bioassay using the EPO dependant UT-7 cell line and the MTT cell proliferation method (Komatsu et al., Blood vol 82, No. 2 (July 15), 1993: pp 456-464). UT-7 cells (human leukemic cell line) are grown in UT-7 Culture Media supplemented with feed EPO to give a final EPO concentration of 1 U/mL. This medium can be prepared by mixing 500 mL of RPMI-1640 media (Gibco catalogue #11875-093 or equivalent), 50 mL of HI-FBS (Gibco catalogue #10438-026 or equivalent), 10 mL of 2.5M 2-mercaptoethanol (174 mL 2-mercaptoethanol, ACS grade or equivalent+826 mL of D-PBS, Gibco cat #14040-026 or equivalent. Sterile filtered using a 0.2 mm syringe filter) in a laminar flow hood. Then sterile filter into a sterile 500 mL bottle using a 0.2 mm bottle-top filter.

Cells should be subcultured or fed every 48-96 hrs. Subculturing concentrations can range from 0.5×10⁵-2×10⁵ cells/mL depending on the length of time before the next subculture or feeding. The cells are to be incubated at 37° C. in a 5% CO₂ atmosphere. On the first day of experimentation, UT-7 cells are deprived of growth factor. Cells are counted to determine cell concentration, with a minimum of 4×104 cells needed per assay well. Cells are removed as needed and placed in a sterile centrifuge tube for centrifugation at 270 g for 5 minutes. The supernatant is then aspirated and the cells are resuspended in half the volume of UT-7 culture media, followed by centrifugation of cells again at 270 g for 5 minutes. The resulting supernatant is aspirated cells are resuspended to a cell seeding concentration of 2×10⁵ cells/mL. Following transfer of the cell suspension to T-75 culture flasks, cells are incubated at 37° C., with 5% CO₂ for 20-26 hrs.

The second experimental day involves preparation of cells for use in the assay and plating of standard and samples. The cells are centrifuged at 270 g for 5 minutes, the supernatant is aspirated, and cells are resuspended in half the volume of assay media. Assay medium is prepared by mixing together 200 ml of 25 mg/mL insulin [insulin (Boehringer Mannheim catalogue #1376497) dissolved in sterile, filtered D-PBS, pH 3.0], 500 mL of RPMI-1640 without phenol red (Gibco cat #11835-030 or equivalent), 200 mL of 25 mg/mL transferrin (Boehringer Mannheim cat #1073974 or equivalent) in D-PBS, 20 mL of 2.5M 2-mercaptoethanol, 1 mL of 10 mg/mL gentamicin (Gibco cat #15710-023 or equivalent). Assay medium can be stored at 2-8° C. The suspension should result in an approximate cell concentration of 4×105 cells/ml. Cells can now be returned to a 37° C., 5% CO2 incubator until needed (up to 3 hrs).

Dilute the EPO standard (R&D Systems Inc. Catalogue #287-TC reconstituted in 0.1% BSA in D-PBS) in the assay medium to a concentration of 20 U/mL, and add 100 mL of the 20 U/mL EPO standard to wells A1, B1, and C1 of a 96 well plate. Add 50 mL of assay media to wells A2, B2, C2 through A12, B12 and C12 inclusive. Serial dilution is performed by removing 50 mL from wells A1, B1, and C1 and transferring it to wells A2, B2, and C2. This series dilution is continued until wells A10, B10, and C10 are reached at which point 50 mL are removed from these wells and discarded. Wells All through C12, inclusive, are blank wells and should contain only 50 mL of assay media at this time.

Samples are prepared by dilution in 0.1% BSA in D-PBS to a concentration at which they can be diluted at least a further 1/10 in assay media prior to addition to the assay plate. 100 μL of sample dilution is added to the sample/unknown plate in triplicate, followed by addition of 50 μL assay media to wells in which the sample will be serially diluted. Serial dilution of the sample is performed by removing 50 μL from the starting wells and transferring this to the next set of wells. This is repeated until appropriate dilution range is obtained and then 50 μL of sample/unknown dilution from the last set of wells is discarded.

UT-7 cells are removed from the incubator and resuspended, followed by transfer of cells to a sterile petri dish. 100 mL of cells is added to all standard and sample/unknown wells, and plates are incubated at 37° C., 5% CO₂ for 44-76 hrs.

Following the incubation, 50 μL of 4.0 mg/mL MTT [3-(4,5-Dimethylthiazolyl-2)-2,5-Diphenyltetrazolium Bromide] (Sigma catalogue #M5655 in 50 ml D-PBS) is added to each standard and sample/unknown well. Plates are agitated on a plate shaker for approximately 30 seconds and then returned to the incubator for 2.5-4 hrs. Plates are centrifuged at 3250 rpm for 15 minutes, and supernatant aspirated. 50 mL of isopropanol is added to all standard and sample/unknown wells, followed by agitation of sealed plates for 5 minutes. Absorbance is read at 595 nm.

The log dose-response curves for the standard and samples are plotted, the 50% of maximum O.D. is calculated, and linear regression is performed. Using the shared slope between the sample and the standard and the y-intercept for the standard curve, the EPO concentration of the standard which corresponds to the 50% O.D. is calculated and the quantity of EPO which results in the 50% O.D. Potency of the sample unknown is then calculated as follows:

$$\frac{EPO ( U ) at 50 \% maximum response O . D from the standard}{EPO ( μg ) of sample at 50 \% maximum response O . D}$$

### EXAMPLE 14

The hGH activity of the hGH RHAMM conjugate can be determined by bioassay using the Nb2 cell line. The Nb2 rat lymphoma cell line was originally derived from a transplantable lymphoma which arose in the lymph nodes of an estrogenized male rat of the Noble (Nb) strain. The growth of Nb2 cells is dependent upon the presence of lactogenic hormones such as prolactin or growth hormone (Gout, P., Beer, C. & Noble, R. 1980. Cancer Research 40: 2433-2436; Tanaka, T., Shiu, R., Gout, P., Beer, C., Noble, R. & Friesen, H. 1980. J. Clin. Endocrin. Metab. 51: 1058-1063).

Standard rhGH is dissolved in purified water to a concentration of 6.0 IU/mL (2 mg/mL). This is further diluted in hGH dilution buffer to a concentration of 400 nIU/mL (133.3 ng/mL). For each two-plate bioassay, dilute this hGH solution further in Fisher's hGH assay medium to a concentration of 8000 nIU/ml.

hGH dilution buffer is prepared by dissolving 2.0 g glycine, 0.2 g mannitol, 0.2 g lactose, and 0.25 sodium bicarbonate in 0.80 ml purified water. Then bovine serum albumin is dissolved to 0.5%, and purified water added to final volume. Fisher's hGH assay medium can be prepared by combining heat-inactivated horse serum (to 10%) and 2-mercaptoethanol (to 0.1 mM) in Fisher's medium. Fisher's medium is a solution of 10% heat-inactivated horse serum (HI-HIS), 10% heat-inactivated fetal bovine (HI-FBS) serum, in Fisher's medium (FM).

The Nb2-11 cells are seeded in Fisher's medium and placed in a 37.0±2.0° C./5.0±0.5% CO2 humidified incubator. Cells must be subcultured before the concentration reaches 1×10⁶ cells/ml to prevent the adaptation of cells to growth in HI-HS alone or development of a heterogeneous hGH growth-dependent cell population (approximately every 72-96 hours).

Nb2-11 cells are suspended in FM at a concentration of 1×10⁵ cells/mL. A growth factor deprivation flask and control are prepared using a volume of cell suspension containing 1×10⁶ cells for preparation of 10 mL at a concentration of 1×10⁵ cells/mL. The flask is then incubated at 37.0±2.0° C./5.0±0.5% CO₂ until the cell count in the control flask plateaus (approximately 96-120 hours).

The cells are centrifuged at 270×g for 5 minutes, washed in assay medium, and recentrifuged. The supernatant is discarded ad the cells are resuspended in assay medium at a concentration of 8×10⁶ cells (8×10⁵ cells/mL). Following incubation at 37.0±2.0° C./5.0±0.5% CO₂ for 24.0±2.0 hours, the cells will be in stationary phase.

During the bioassay, blank wells contain 100 μL of FhAM+100 μL of cells+tracer and are used to monitor the uptake of tracer (i.e. cell proliferation) in the absence of rhGH and to provide the minimum response value for calculation of the 50% maximal response.

Serial dilutions are made of rhGH standard and samples following determination of sample protein concentration. The contents of the stationary flasks are pooled, and cells counted. Cell concentration is adjusted to 4×10⁵ cells/mL by the addition of assay medium. A volume of 20 mL is normally prepared for each bioassay plate to ensure an excess of cell suspension. This volume can be reduced to a minimum of 12.0 mL per plate if there is a limiting number of cells. The prepared cells are transferred into a tissue culture dish at a maximum volume of 40 mL per dish and mixed. 100 mL of the cells are added at a concentration of 4×10⁵ cells/mL (4×10⁴ cells/well) to all wells taking care to mix the cells prior to each uptake by gently stirring. The plates are incubated at 37.0±2.0° C./5.0±0.5% CO₂ for 70 hours.

Cells are labelled with 20 mL of tracer (1/100 ³H-methyl-thymidine in assay medium) in each well, followed by incubation for 2.0±0.25 hours at 37.0±2.0° C./5.0±0.5% CO₂.

Cells are harvested from the plates onto filter discs, washed, and placed into scintillation vials with 5 ml of scintillation fluid for counting. A regression analysis is performed on the standard and samples, which will be compared to determine parallelism. The potency of the sample can be determined by the following equation:

$$\frac{{EC}_{50}^{std} ( nIU \text{/} ml )}{concentration of sample aliquot with same response as {EC}_{50}^{std} ( pg \text{/} ml )}$$

### EXAMPLE 15

To investigate the effect of the RHAMM-hGH conjugate on body weight gain, hypophysectomized male rats will undergo daily subcutaneous injection for a minimum of 10 consecutive days. Male Sprague-Dawley hypophysectomized rats (6 weeks of age, 70-90 g) will be purchased and shipped to the animal care facility. Following arrival, each animal will be given a general physical examination by a member of the veterinary staff to assess health status. An acclimation period of approximately 2 weeks will be allowed between animal receipt and the start of treatment in order to accustom the animals to the laboratory environment. Animals will be housed individually at 22±3° C. with a 12 hour light/dark cycle in stainless steel wire mesh-bottomed cages equipped with an automatic watering valve. Municipal tap water which has been softened, purified by reverse osmosis and sterilized by ultraviolet light will be freely available (except during designated procedures). All animals will have free access to a standard certified pelleted commercial laboratory diet (PMI Certified Rodent Chow 5002: PMI Feeds Inc.) except during designated procedures.

Approximately 1 week before treatment initiation, all animals will be weighed and assigned to treatment groups using a computer generated randomization procedure. There will be 7 treatment groups each with 16 animals, consisting of one control group (0.9% saline solution), three groups receiving hGH at doses of 10, 50, and 100 μg/rat/day, and three groups receiving the RHAMM-hGH conjugate at doses to be determined based on preliminary studies. The intrascapular injections will be made in a volume of 1 mL, and shall be administered once daily for 9 consecutive days. During the first 5 days of treatment, animals will have free access to a water bottle containing 5% Dextrose, U.S.P. During the last 4 days of treatment, the solution will be reduced to a concentration of 2.5% Dextrose, U.S.P.

All animals will be examined twice daily for mortality and signs of ill-health or reaction to treatment. Death and observed clinical signs will be individually recorded. Individual body weights will be measured daily for at least 10 days prior to treatment and continuing throughout the treatment period. Following 10 days of treatment (i.e. on day 11), the animals will be euthanized by carbon dioxide asphyxiation and the carcasses discarded without further examination.

The osteogenic effects of RHAMM-protein conjugates (eg. RHAMM-hGH, RHAMM-PTH) must be determined in vivo. The RHAMM-protein conjugate or inactive protein conjugate will be administered to 2 groups of rats, respectively, at dose schedules and intervals to be determined by preliminary studies (5 days/week for 2 months). Peripheral quantitative computed tomography (pQCT) will be used to measure bone mineral content, bone density, and geometric parameters of the right distal femur ex vivo. Femur was selected since it is easily scanned and is relatively straight, and increases in bone mineral content and bone area following GH treatment have been documented in femur by this method (Banu, J., Orhii, P. B., Okafor, M. C., et al., 1999). In addition, parathyroid hormone has also been shown to increase femoral bone mineral density and bone area, and the combination of GH and PTH increases bone strength more than either therapy alone (Wang, L., Orhii, P. B., Banu, J., and Kalu, D. N., 1999).

pQCT scans will be performed using a Norland XCT Research SA bone scanner with software version 5.40. A single scan will be obtained of the femoral metaphysis at approximately 18% of the bone length as measured from the distal end of the femur. In addition, a second scan will be acquired at 50% of the bone length. The pQCT procedure will characterise any changes in bone geometry and bone density due to treatment. Distal femoral scans will be evaluated for area, bone mineral content, and bone density of the total slice and the trabecular and cortical/subcortical regions, periosteal circumference and endosteal circumference.

In addition to these endpoint parameters, biochemical markers of bone turnover will be analyzed pre- and post-treatment, such as osteocalcin, bone-specific alkaline phosphatase, and PTH in blood, and deoxypyridinoline, N-telopeptide, and creatinine in urine.

### EXAMPLE 16

Production of RHAMM-GMCSF Direct Conjugate using Backbone Engineered Thioether Linkage

GMCSF would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated GM-CSF to a RHAMM peptide will increase the half-life of GM-CSF and direct the conjugate to tissues with a high concentration of HA. It has been reported that approximately 25% of HA is found in bone, which is an optimal target for GM-CSF, and as such, in addition to increasing half-life, conjugation to RHAMM may enhance target specificity.

Preparation of Recombinant GMCSF

The complete coding DNA sequence of human GM-CSF is given as GenBank Accession No. E00951. Recombinant GM-CSF may be purchased from existing commercial sources, or produced by methods known to persons skilled in the art.

A iodoacetyl group must be added to the N-terminal of GMCSF for attachment of the RHAMM peptide. This may be accomplished by adjusting a GMCSF solution (originally in 10 mM phosphate, pH 6.5) to 60 mM phosphate buffer and 0.15% pluronic at a pH of 6.0. Iodoacetic anhydride is added to a final 10-fold molar excess, i.e., an equimolar quantity is added in 10 aliqouts at 5 minute intervals at room temperature. Following iodoacetylation, the protein is purified by gel filtration on a PP10 coulumn. The preparation of recombinant GMCSF for thioether linkage conjugation to RHAMM is illustrated structurally below, where H2N— and —OH represent the N and C terminals of GMCSF, respectively.

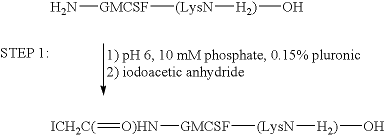

Preparation of RHAMM Peptide

SEQ. I.D. 3 comprises the preferred sequence of RHAMM peptide for conjugation to EPO. The RHAMM peptide must be engineered with an C-terminal Cysteine group, which will later be ligated to the iodoacetylated N-terminal. To this end, a benzhydrylamine resin is utilized to produce a C-terminal amide and the first residue coupled to the resin is the amino acid Cysteine. RHAMM is then synthesized as per normal Boc or Fmoc solid phase peptide synthesis (SPPS) methods. Finally, SEC and/or RP-HPLC methods are used to purify the peptide. The process is illustrated structurally below.

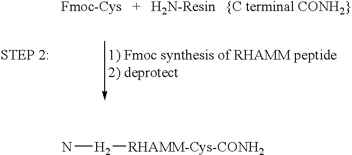

Coupling of the RHAMM Peptide and GMCSF

Iodoacetylated GMCSF is buffer exchanged into 10 mM phosphate,. pH 7.0. The RHAMM-Cys peptide is reconstituted in 10 mM phosphate, pH 7.0 and added to the Iodoacetylated GMCSF solution. The solution is allowed to incubate at room temperature for 30 minutes (reference if any). Purification of the conjugate is by performed by RP-HPLC followed by SEC. The structural representation of the conjugation reaction is illustrated below.

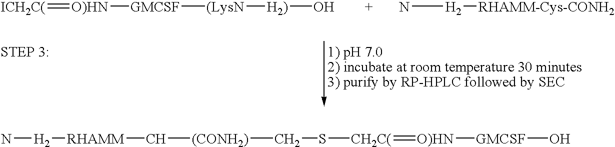

Characterization

The level of conjugation can be assessed by determining the mass and amino acid composition of the conjugate and a tryptic digestion map can verify the conjugation site.

Diagramatic Representation of Reaction Steps

1. Modify protein with sulfo-GMBS:

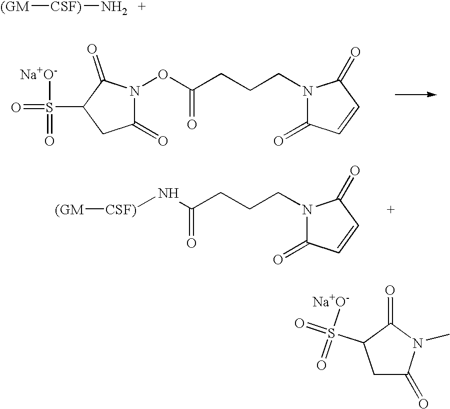

2. Synthesize peptide with SAMA N-terminal:

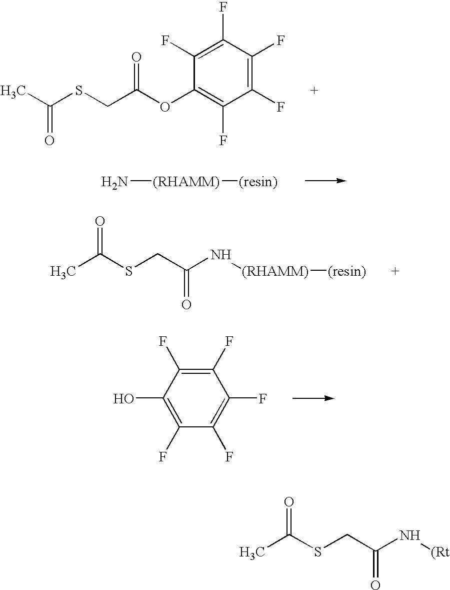

3. Deprotect peptide with hydroxylamine:

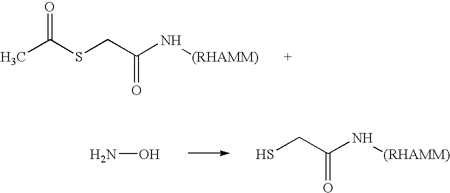

4. Link modified protein to deprotected peptide:

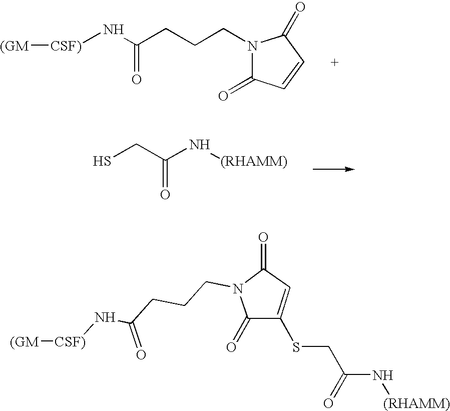

Characterization of RHAMM-GM-CSF Conjugate

The following describes the amino acid analysis of peptides and proteins using precolumn derivatization with PITC to form the PTC amino acids.

Materials: Chemicals

Constant boiling 6N hydrochloric acid (sequenal grade) (Pierce/Chromatographic Specialties Cat. #24309)

Amino acid standard H, 2.5 μmole/mL (Pierce/Chromatographic Specialties Cat. #20088)

Type I reagent grade purified water or equivalent

Methanol (HPLC grade) (Caledon Cat. #6701-7 or equivalent)

Phenylisothiocyanate, 1.13 g/mL (FW=135.2) (sequenal grade) (Pierce/Chromatographic Specialties Cat. #26922)

Triethylamine, 10×2 mL ampoules (sequenal grade) (Fluka Cat. #90338; distributed through Sigma Chemical Co.)

Glacial acetic acid (ACS reagent) (Fisher Scientific Co. Cat. #A38-4 or equivalent)

Sodium acetate, anhydrous (NaC₂H₃O₂, FW=82.03) (ACS reagent) (Sigma Chemical Co. Cat. #S-5889 or equivalent)

Sodium phosphate dibasic heptahydrate (Na₂HPO₄.7H₂O, FW=268.07) (ACS grade) (BDH Cat. #ACS810 or equivalent)

Acetonitrile-190 (CH₃CN) (HPLC grade) (Caledon Cat. #1401-7 or equivalent)

Prepurified nitrogen (T-size and lecture bottle size) (Medigas Cat. #105 395)

Phenol (C₆H₆O) (SigmaUltra) (Sigma Chemical Co. Cat. #P-5566 or equivalent)

S-Carboxymethyl-_{L}-cysteine (FW=179.2) (Sigma Chemical Co. Cat. #C-7757)

O-phosphoric acid, 85% (HPLC grade) (Fisher Scientific Co. Cat. #A260-500 or equivalent)

Standard peptide, Ac-RGVGGLGLGK-amide; 0.069 mg/vial or equivalent concentration by AAA (API)

Materials: Equipment

Pico-Tag work station (Waters Chromatography Division Cat. #07370)

Refrigerated condensation trap (RT100) (Fisher Scientific Co.)

Microfuge (Eppendorf or equivalent)

Ultrasonic bath #FS9 (Fisher Scientific Company Cat. #15-336-2 or equivalent)

Thermolyne #1400 furnace (Fisher Scientific Company Cat. #10-552)

Vacuum pump, Leybold D2A (Fisher Scientific Company Cat. #01-058-6 or equivalent)

Gilson pipetman: P20 (20 μL) (Mandel Cat. #GF-23600 or equivalent), P200 (200 μL) (Mandel Cat. #GF-23601 or equivalent), P1000 (1000 μL) (Mandel Cat. #GF-23602 or equivalent)

Disposable glass culture tubes (16×100 mm) (Fisher Scientific Company Cat. #14-961-29)

Disposable glass culture tubes (12×75 mm) (Fisher Scientific Company Cat. #14-961-26)

Pyrex culture tubes with screw cap (16×100 mm) (Fisher Scientific Company Cat. #14-930-13B)

Screw cap for 4.2.10 with teflon inserts (Fisher Scientific Company Cat. #14-930-15E)

Graduated glass cylinder (500 mL) (Fisher Scientific Company Cat. #08-549-5H or equivalent)

Microfuge vials, 1.5 mL (InterScience Cat. #QS509GRDY or equivalent)

Amino acid hydrolysis tubes (culture tubes, 6×50 mm) (Waters Chromatography Division Cat. #WAT007571)

2 reaction vials with 2 reaction vial caps (Waters Chromatography Division Cat. #7363)

200 μL Microcapillary tips, polypropylene (Sigma Chemical Co. Cat. #T-1906)

Filter paper (0.45 μm or smaller) (Millipore Corporation Cat. #HVLP04700 or equivalent)

Acrodisc Filter, 0.2 μm (Gelman Sciences Part #4192)

HPLC System

Beckman HPLC System containing the following modules: #507e autosampler, #166 programmable detector module, #126 programmable solvent module computer equipped with System Gold Nouveau software, CIN 2714 (Beckman Instruments Inc.)

Target vials (Beckman Cat. #887954, National Scientific Company Cat. #C4011-1)

Target vial septa (Beckman Cat. #C40041, National Scientific Company Cat. #C4010-60)

Target vial insert: low volume insert, 300 μL (Beckman Cat. #C40042, National Scientific Company Cat. #C4010-630)

Injection loops: 20 μL for Rheodyne #7725 injector (Beckman Cat. #890870)

Back-pressure unit, 100 psi (Chromatographic Specialties Inc. Cat. #M100600130 or equivalent)

Biorad column heater (Biorad Cat. #125-0425)

Reversed-phase column: Pico-Tag column (Waters Cat. #WAT088131)

Pre-column filter, 1/16″, PEEK, 2 μm frit (Supelco Cat. #Z-227323)

Replacement filters for column pre-filter, 2 μm (Sigma-Aldrich Cat. #Z22,733-1)

Order of Events

Day 1=Dry down samples and hydrolyse samples overnight.

Day 2=Dry down standards, sample hydrolysates and derivatization blank.

Day 2=Derivatize samples, standards and derivatization blank.

Use of Refrigerated Trap

If the trap has been used to dry down samples that contain HCl, exchange the trap for a clean trap before drying down samples containing PITC (i.e., during the derivatization incubation, a clean trap should be installed).

Preparation of Amino Acid Stock Solutions

Preparation of 10 mM HCl:

Transfer 6N HCl from ampoule to an amino acid clean screw cap culture tube.

Prepare 10 mM HCl by mixing 15 μL (6N HCl)+8985 μL purified water.

Store in a screw cap culture tube at 2-8 C.; expiry=6 months

Preparation of Amino Acid Standard:

Amino acid stock solution #1: Transfer amino acid standard H (2.5 μmole/mL each amino acid) to an amino acid clean screw cap culture tube. Prepare amino acid stock solution #2 by mixing 100 μL amino acid standard H (2.5 μmole/mL)+400 μL 10 mM HCl and store in a screw cap culture tube. This represents 6 nmole of each amino acid/12 μL solution. (2.5 μmole/1000 μL) (100 μL)=0.25 μmole 0.25 μmole/500 μL=0.0005 μmole/μL) (0.0005 μmole/μL) (12 μL)=0.006 μmole=6 nmole).

Preparation of CMC Standards:

CMC stock solution #1: Dissolve 20 mg (catch weight) of CMC in 5.0 mL of 10 mM HCl to yield 0.0223 mmole CMC/mL. (Note: Adjust the volume of 10 mM HCl to the actual weight of CMC to yield 0.0223 mmole CMC/mL). 20 mg/179.2=0.112 mmole/5 mL=0.0224 mmole/mL

CMC stock solution #2: Add 50 μL stock solution #1 to 450 μL of 10 mM HCl. (0.0223 mmole/1000 μL)×50 μL=0.001115 mmole (0.001115 mmole/500 μL)×(10⁶ nmole/mmole)=2.23 nmole/μL

CMC stock solution #3: Add 70 μL stock solution #2 to 240 μL 10 mM HCl to yield 0.5 nmole CMC/μL. (70 μL) (2.23 nmole/μL)=156 nmole; 156 nmole/310 μL=0.5 nmole/μL

Preparation of Dried Standard for Derivatization

A volume of 20 μL reagent/sample is sufficient to derivatize 12.5 nmole of each amino acid or a total of 250 nmole amino acid [There is a total of 19-amino groups×12.5 nmole=237.5 nmole total 250 nmole of amino acid]. Dispense 12 μL (6 nmole each amino acid) of standard solution #2 into each of 4 amino acid clean hydrolysis tubes (6×50 mm). Dispense 12 μL of 10 mM HCl into an amino acid clean hydrolysis tube for the derivatization blank. Add 6 nmole of CMC to each of the standards and derivatization blank. Dry down samples on Pico-Tag station: Pressurize reaction vial with nitrogen. Close nitrogen valve. Open vacuum valve and allow to dry until vacuum reaches approximately 65 mTorr. Close vacuum valve. Pressurize reaction vial with nitrogen.

Sample Digestion

Dispense approximately 0.3 nmole of protein or approximately 10 nmole of a peptide into each of at least 4 amino acid clean hydrolysis tubes (6×50 mm). This value is a starting point. If the composition is known, hydrolyse enough sample so that there is approximately 0.2-3.0 nmole each amino acid per 20 μL load. Once the samples have been analysed, the quantities loaded may have to be adjusted to obtain the suggested 0.2-3.0 range.

Target=1-3 nmole most abundant amino acid/20 μL (as in standard curve). The derivatives will be dissolved in 120 μL and 20 μL will be loaded onto the column; Dilution=120/20=6. Therefore, 6-18 nmole of the most abundant amino acid required. Most abundant AA: Target=(1-3 nmole most abundant AA/20 μL):

(#nmole most abundant AA)/(#nmole most abundant AA/nmole protein)=nmole protein required

(#nmole protein)(molecular weight of protein in ng/nmole)/(1000 ng/μg)=μg protein required

(μg protein)/(concentration of protein in μg/μL)=μL protein required

Check the Least Abundant AA:

(#nmole protein)(#nmole least abundant AA/nmole protein)=nmole least abundant AA

(#nmole least abundant AA)(20/120)=nmole least abundant AA present (accounting for dilution of sample)

Ensure this value does not exceed the low limit (i.e., 0.2 nmole).

Determine the total number of nmole of amino acid being derivatized, i.e., 1 nmole of protein or peptide=x nmole of amino acid, where x=the number of residues.

For example, 1 nmole of hGH (191 amino acids)=191 nmole amino acid Therefore, 0.3×191=57.3 nmole amino acid <250 nmole as stated above.

Place in reaction vial and dry down on Pico-Tag station as above. Add 200 μL 6N HCl to the bottom of the reaction vial. Add one crystal of phenol to the bottom of the reaction vial. Perform a total of 3 vacuum-nitrogen flush cycles [vacuum 1-2 torr and nitrogen 5 second flush].

Ensure that nitrogen and vacuum valves are closed. Place reaction vial on the Pico-Tag station. Open nitrogen valve for approximately 5 seconds; close nitrogen valve. Open vacuum valve until vacuum is approximately 1-2 torr (HCl should just start to evaporate); close vacuum valve. Repeat twice. Check bottom of reaction vial to ensure that there is still HCl present.

Place reaction vial into oven at 110±1 C. for 24 hours. After the hydrolysis time period, remove the reaction vial and allow to cool at room temperature (˜10-15 minutes). Transfer hydrolysis tubes to a second reaction vial. Dry down samples on Pico-Tag station as above. Add 6 nmole of CMC per sample (i.e., 12 μL of CMC stock solution #3) and dry down samples on Pico-Tag station as in 6.4.4.

Preparation of Redry/Derivatization Reagents

In a 12×75 mm culture tube, prepare redrying reagent (methanol/water/TEA, 2:2:1) as follows: 100 μL methanol, 100 μL water, 50 μL TEA, 10 μL/sample=25 samples.

In a 12×75 mm culture tube, prepare derivatization reagent (methanol/TEA/water/PITC, 7:1:1:1) as follows: 210 μL methanol, 30 μL TEA, 30 μL water, 30 μL PITC, 20 μL/sample=15 samples.

Derivatization of Standards and Samples

Redry step: Add 10 μL redrying reagent to each tube and mix. Dry down standards, samples and blank on Pico-Tag station as above.

Derivatization step: Add 20 μL derivatization reagent to each tube and mix. Allow to incubate at room temperature (20-25 C.) for 20 min. Dry down standards, samples and blank on Pico-Tag station as above.

Preparation of HPLC Reagents

Solvent A (140 mM acetate, pH 6.4/ACN [94/6, v/v]):

11.5 g sodium acetate+950 mL purified water+500 μL TEA. Adjust pH to 6.4 with glacial acetic acid. Adjust volume to 1000 mL and filter. Dispense 893 mL buffer+57 mL ACN.

Solvent B (60% ACN/water):

360 mL purified water+540 mL ACN

Sample Diluent (5 mM Na₂HPO₄, pH 7.4/ACN [95/5, v/v]):

0.13 g Na₂HPO₄.7H₂O in 90 mL of purified water. Adjust to pH 7.4 with 5% H₃PO₄.5% H₃PO₄=5.9 mL H₃PO₄ (85%) up to 100 mL with PW. Make final volume to 100 mL with PW and filter through 0.2 μm. Mix 95 mL of the filtered buffer+5.0 mL ACN using a stirrer.

20% MeOH (Storage solvent for unused lines):

Add 200 mL methanol to 800 mL purified water using a graduated glass cylinder.

Column Installation and Equilibration

Install column with PEEK tubing (0.010″ I.D., blue) at the inlet and outlet and a 100 psi back pressure regulator at the detector outlet. Load reagents as follows:

TABLE 1

| Reagent | Line |
|---|---|
| Solvent A (140 mM sodium acetate, pH 6.4/ | A1 (Red) |
| ACN [94/6, v/v]) | |
| Solvent B (60% ACN/purified water) | B1 (Red) |
| Purified water (degassed) | A2, B2 (Yellow) |

Prime each line as per HPLC SOP #84.030.0004.RR. Prime pumps with wastevalve open, selector valve set at (A1, B1), and pumps set at 50% B [50% A]. Set the flow rate values onscreen to 3.000, 0.00 min. Wash the back side of the pistons of the HPLC pumps with water. Set the flow rate values to 0.000, 0.00 min. Close the drain valve by turning it clockwise until tight.

Set upper pump pressure limit to 2000 psi and lower pump pressure to 0.05 psi. Under DIRECT CONTROL, set pump to 100% B [0% A], (A1, B1). With waste valve closed, set pump flow to 1 mL/min in 1 min. When pump has reached 1 mL/min, set 100% B [0% A] to 0% B [100% A] in 5 min. with selector valve at (A1, B1). After 5 min period, wash column with buffer A (to remove storage solvent) for 15 min. (at this point the baseline should be stable).

HPLC Conditions

Experimental conditions:

Flow rate=1.0 mL/min

Temperature=38 C.

Monitoring wavelength=254 nm

Cooling tray=6 C.

Injection mode=Regular (Full loop requires a minimum of 100 μL sample.)

TABLE 2
Gradient table:

| Time (min) | % A | % B | Duration (min) | Curve (CV) |
|---|---|---|---|---|
| INITIAL | 100 | 0 | — | — |
| 0 | 58 | 46 | 10 | 3 |
| 10 | 0 | 100 | 0.5 | 0 |
| 10.5 | 0 | 100 | 5.5 | 0 |
| 16 | 100 | 0 | 1 | 0 |
| 17 | 100 | 0 | 8 | 0 |

Equilibration requires 20 mL of Eluent A and 5 mL of Solvent B: each run requires ˜20 mL Solvent A and ˜12 mL Solvent B.

Dissolution of Standards and Samples

Dissolve each of the derivatized standards (6 nmole each amino acid) and each of the derivatized samples (1.6-16 nmole each amino acid) in 120 μL sample diluent. Centrifuge each of the standards and samples at 13 000 rpm for 2 minutes. Load 20 μL of standard onto column, which is equivalent to 1 nmole each amino acid/20 μL load. Evaluate the standard curve, i.e., determine if all of the peaks have been identified as indicated in table 7.1. If all peaks can not be accounted for, prepare new buffers and re-run the standard curve. If peak identification is still a problem, then the HPLC may require maintenance. Refer to the SOP entitled “Calibration Testing of the Beckman HPLCs”. Load 20 μL of sample onto column, which is equivalent to 0.27-2.7 nmole each amino acid/20 μL load. 1.6/6=0.27 nmole each AA; 16/6=2.7 nmole each AA.

Column Storage

Following completion of the shutdown method (see Exhibit 3), remove lines A1 and B1 prime lines with PW. Place both lines in 20% methanol/water and prime lines. Open waste valve. Before starting pumps, flush back side of pumps with PW. With selector valve set at 50% B [50% A], set the flow to 3.000 mL/min in 0.000 min and continue to flush the back side of the pumps with up to 60 mL PW. Close waste valve.

Data Aanalysis

The elution order of the PTCaa is shown in the following table:

TABLE 3

| Peak # | Amino acid |
|---|---|
| 1 | D (Asp) |
| 2 | E (Glu) |
| 3 | CMC |
| 4 | S (Ser) |
| 5 | G (Gly) |
| 6 | H (His) |
| 7 | R (Arg) |
| 8 | T (Thr) |
| 9 | A (Ala) |
| 10 | P (Pro) |
| 11 | Y (Tyr) |
| 12 | V (Val) |
| 13 | M (Met) not used |
| 14 | C (Cys) not used |
| 15 | I (Ile) |
| 16 | L (Leu) |
| 17 | F (Phe) |
| 18 | K (Lys) |

Results

N—N conjugate: The amino acid composition of the conjugate matches the expected amino acid sequence of composition of GMCSF+RHAMM indicating successful N—N conjugation.

TABLE 4

| | GMCSF control | | | RHAMM^{N}-^{N}GMCSF conjugate | | |
|---|---|---|---|---|---|---|
| | Deter- | | | Deter- | | |
| | mined | Integer | Expected | mined | Integer | Expected |
| AA | value | value | value | value | value | value |
| G | 3.02 | 3 | 3 | 3.10 | 3 | 3 |
| H | 6.03 | 6 | 6 | 2.93 | 3 | 3 |
| R | 8.28 | 8 | 8 | 5.78 | 6 | 6 |
| V | 5.87 | 6 | 6 | 4.95 | 5 | 5 |
| F | 4.97 | 5 | 5 | 5.18 | 5 | 5 |
| K | 6.83 | 7 | 7 | 6.05 | 6 | 6 |

C—N conjugate: The amino acid composition of single and double conjugate matches the expected composition of GMCSF+RHAMM and GMCSF+2×RHAMM, respectively indicating successful C—N conjugation.

TABLE 5

| | RHAMM^{C}-^{N}GMCSF conjugate | | | RHAMM₂^{C}^{_{-N}}GMCSF conjugate | | |
|---|---|---|---|---|---|---|
| | Deter- | | | Deter- | | |
| | mined | Integer | Expected | mined | Integer | Expected |
| AA | value | value | value | value | value | value |
| G | 3.13 | 3 | 3 | 3.23 | 3 | 3 |
| H | 5.86 | 6 | 6 | 8.73 | 9 | 9 |
| R | 7.95 | 8 | 8 | 10.34 | 10 | 10 |
| V | 5.74 | 6 | 6 | 6.68 | 7 | 7 |
| L | 13.91 | 14 | 14 | 14.16 | 14 | 14 |
| F | 5.08 | 5 | 5 | 5.05 | 5 | 5 |
| K | 7.33 | 7 | 7 | 7.81 | 8 | 8 |

Demonstration of Bioactivity of RHAMM-GM-CSF Conjugate:

The following describes the procedures for an in vitro bioassay to determine the biological activity of LEUCOTROPIN™ (GM-CSF, Granulocyte-Macrophage Colony Stimulating Factor) using the TF-1 cell line and the MTT cell proliferation method. Materials required include the following:

Materials:

TF-1. Cells: Human derived cell line of immature erythroid origin that proliferates dependently on GM-CSF.

Dulbecco's phosphate buffered saline (D-PBS): Gibco cat #. 14040-026 or equivalent. Manufacturer's expiry date.

2.5M 2-mercaptoethanol (1 mL): Mix 174 μL .2-mercaptoethanol, ACS grade, 826 μL D-PBS and sterile filter using a 3 mL syringe and 0.22 μm syringe filter. Culture media: 500 mL DMEM, Gibco cat #11965-050 or equivalent, 50 mL

Heat-Inactivated Fetal Bovine Serum (HI-FBS) from a certified and reliable source, 10 μL 2.5M 2-mercaptoethanol

Bovine serum albumin (BSA), fraction V, Gibco cat #11018-041 or equivalent

D-PBS+0.1% BSA (sterile): 100 mL D-PBS, 0.1 g BSA, Dissolve BSA in D-PBS and sterile filter with 0.22 μm filter.

4.0 mg/mL MTT stock solution (sterile), 50 mL: 0.2 g MTT, Sigma cat #M5655 or equivalent, 50 mL D-PBS, Dissolve the 0.2 g of MTT in 50 mL of D-PBS and filter sterilize with 0.22 μm filter. Store at 2-8° C., protect from light.

Assay media: 500 mL of RPMI-1640 without phenol red, Gibco cat #11835-030 or equivalent. 200 μL of 25 mg/mL insulin. 100 mg sterile lyophilized insulin, Boehringer Mannheim cat #1 376 497 or equivalent. 2 D-PBS (adjusted to pH 3.0 with HCl, ACS grade or equivalent and sterile filtered)

Dissolve 100 mg insulin (contents of one sterile lyophilized vial) in 4 mL of sterile D-PBS (pH 3.0). Aliquot in 200 μL aliquots and store at −10° C. until needed. 200 μL of 25 mg/mL transferrin. 100 mg lyophilized transferrin, Boehringer Mannheim cat #1 073 974 or equivalent. D-PBS: Dissolve 100 mg transferrin (contents of one sterile lyophilized vial) in 4 mL D-PBS. Aliquot in 200 μL aliquots and store at −10° C. until needed. 20 μL of 2.5M 2-mercaptoethanol. 1 mL of 10 mg/mL gentamicin, Gibco cat #15710-023 or equivalent. Mix above (4.8.1 to 4.8.5), store at 2-8° C.

Cell Preparation

4×10⁴ cells are needed/assay well, or 3.84×10⁶ cells/96 well plate. Remove the volume of cells needed as calculated in 6.1.2 and place them in a sterile centrifuge tube for centrifugation at 270×g for 5 minutes. Aspirate the supernatant and resuspend the cells in _volume of culture media (4.4). Centrifuge cells again as per above. Aspirate the supernatant and resuspend cells in 30 mL of culture media/96 well assay plate required (this will result in a cell seeding concentration of 2×10⁵ cells/mL). Transfer cell suspension to T-75 culture flask(s), 30 mL/flask and incubate at 37° C., 8% CO₂ for 20-24 hours. On day zero of the bioassay, determine the viable cell count of the growth factor-deprived cells. Transfer the volume of cells needed for the assay (Section 6.2.2) to a sterile tube for centrifugation at 270×g for 5 minutes. Aspirate the supernatant and resuspend the cell pellet in _volume of fresh assay medium (4.8). Centrifuge the tube again. Aspirate the supernatant and resuspend the pellet in assay medium. The volume to be added is determined by the following equation: Volume of media (mL)=total # of cells/4×10⁵ cell/mL. 0.1 mL of cell suspension is added/per well at a concentration of 4×10⁵ cells/mL resulting in 4×10⁴ cells/well. The resuspended cells can be incubated after resuspension in the centrifuge tube at 37° C., 5% CO₂ for up to 6 hours until use. Dilute the 20 μL of working stock standard (4.9, 10,000 IU/mL), 16.67× by adding 313.3 μL of assay medium, giving a final volume of 333.3 μL and a concentration of 600 IU/mL. Aliquot 100 μL of the 600 IU/mL standard in triplicate to the first three wells designated for the standard curve on the plate. Add 50 μL of assay medium to all the other wells designated for the standard curve on the plate. Using a 8-channel pipettor with 3 tips attached remove 50 μL from the 100 μL of 600 IU/mL standard wells (6.4.2), and transfer the volume to the next set of designated standard wells containing 50 μL of assay medium. Aspirate well contents up and down five times to ensure proper mixing. This results in a two-fold dilution (i.e 600 IU/mL to 300 IU/mL). Remove 50 μL from this dilution and transfer it to the next set of standard wells containing 50 μL of assay media. Continue this until the tenth dilution, where 50 μL is then removed from the last set of standard wells and discarded. This results in equal volumes in all the standard wells.

Preparation of GM-CSF Test Sample

Each test sample is prepared and diluted resulting in an approximate GM-CSF concentration of approximately 10 ng/μL.

Dilution of GM-CSF Samples for Dose-Response Curves

Add 490 μL of assay medium to the 10 μL aliquot of 10 ng/μL test sample from 6.5 to give a concentration of 200 pg/μL. Remove 70 μL of the 200 pg/μL dilution and further dilute it to 40 pg/μL by adding 280 μL of assay medium. Aliquot 50 μL of assay medium to all the wells to be used for the sample dilutions, except for the first triplicate set. Aliquot 100 μL of the 40 pg/μL starting sample dilution in triplicate to the empty starting sample dilution wells. Transfer in triplicate, using a multichannel pipettor, 50 μL of the starting dilution to the next set of triplicate wells containing 50 μL of assay medium. Aspirate well contents up and down five times to ensure proper mixing. Continue this 2-fold serial dilution until eight dilutions have been made. Remove 50 μL from the last dilution so that they all have the same volume.

Assay Procedures

Add 50 μL of assay medium to 6 wells to act as blanks. Mix the cell suspension from above and add it to a sterile reservoir for dispensing. Add 100 μL of the cell suspension (approximately 4×10⁴ cells/100 μL) to all plate wells containing 50 μL of either standards, blanks or test samples, using the multi-channel pipette. When adding cells be careful not to dip the pipette tips into the wells too far so as not to contact the standard/sample inside which would result in transfer of some of it to other standards/samples. Incubate the assay plate at 37° C., 5% CO₂ for 72±5 hours.

Procedures for Day Three of the Bioassay

After incubation add 50 μL of the 4 mg/mL MTT solution to each well using an 8-channel pipette. Cover the plate with plate sealer and mix for approximately 30 seconds with the plate shaker. Remove plate sealer and incubate the plate for 3±0.5 hours at 37° C., 5% CO₂. Remove plate and centrifuge at 1775×g for 15 minutes, with brake on high. Supernatant

Removal Using Microplate Washer.

Prior to aspirating assay plates check proper functioning of the plate washer by aspirating a blank plate filled with water only. Be sure that all 8 channels are aspirating efficiently and that none are plugged. Aspirate supernatant from all assay plates, using the microplate washer. Add isopropanol, 50 μL/well using a reagent reservoir and multi-channel pipette. Cover the plate with plate sealer and mix for 5 minutes using the plate shaker. Remove plate sealer and read the plates at 595 nm using the microplate reader.

Results

n-b conjugate: mean potency is approximately 50% compared to native GMCSF.

TABLE 6

| | | % potency compared to |
|---|---|---|
| | Mean potency (IU/mg) | control |
| GMCSF control | 1.24 × 10⁷ | 100 |
| RHAMM^{N}^{₋}^{N}GMCSF | 0.57 × 10⁷ | 46 |
| conjugate | | |

C—N conjugate: mean potency is approximately 50% compared to native GMCSF for a single conjugate, and approximately 40% compared to native GMCSF for a double conjugate.

TABLE 7

| | | % potency compared to |
|---|---|---|
| | Mean potency (IU/mg) | control |
| GMCSF control | 1.23 × 10⁷ | 100 |
| RHAMM^{C}^{₋}^{N}GMCSF | 0.66 × 10⁷ | 54 |
| conjugate | | |
| RHAMM₂^{C}^{₋}^{N}GMCSF | 0.48 × 10⁷ | 39 |
| conjugate | | |

### EXAMPLE 17

Production of RHAMM-EPO Direct Conjugate using Backbone Engineered Thioether Linkage

Erythropoietin would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated EPO to a RHAMM peptide will increase the half-life of EPO and direct the conjugate to tissues with a high concentration of HA. It has been reported that approximately 25% of HA is found in bone, which is an optimal target for EPO, and as such, in addition to increasing half-life, conjugation to RHAMM may enhance target specificity.

Preparation of Recombinant EPO

The complete coding DNA sequence of human erythropoietin was first disclosed in U.S. patent application Ser. No. 655,841, filed Sep. 28, 1984, which was later issued as U.S. Pat. No. 4,703,008

A iodoacetyl group must be added to the N-terminal of EPO for attachment of the RHAMM peptide. This may be accomplished by adjusting a EPO solution (originally in 10 mM phosphate, pH 6.5) to 60 mM phosphate buffer and 0.15% pluronic at a pH of 6.0. Iodoacetic anhydride is added to a final 10-fold molar excess, i.e., an equimolar quantity is added in 10 aliqouts at 5 minute intervals at room temperature. Following iodoacetylation, the protein is purified by gel filtration on a PP10 coulumn. The preparation of recombinant EPO for thioether linkage conjugation to RHAMM is illustrated structurally below, where H2N— and —OH represent the N and C terminals of EPO, respectively.

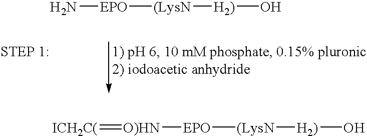

Preparation of RHAMM Peptide

SEQ. I.D. 3 comprises the preferred sequence of RHAMM peptide for conjugation to EPO. The RHAMM peptide must be engineered with an C-terminal Cysteine group, which will later be ligated to the iodoacetylated N-terminal. To this end, a benzhydrylamine resin is utilized to produce a C-terminal amide and the first residue coupled to the resin is the amino acid Cysteine. RHAMM is then synthesized as per normal Boc or Fmoc solid phase peptide synthesis (SPPS) methods. Finally, SEC and/or RP-HPLC methods are used to purify the peptide. The process is illustrated structurally below.

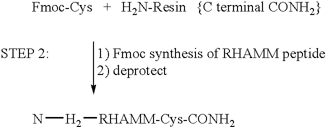

Coupling of the RHAMM Peptide and EPO

Iodoacetylated EPO is buffer exchanged into 10 mM phosphate, pH 7.0. The RHAMM-Cys peptide is reconstituted in 10 mM phosphate, pH 7.0 and added to the Iodoacetylated EPO solution. The solution is allowed to incubate at room temperature for 30 minutes (reference if any). Purification of the conjugate is by performed by RP-HPLC followed by SEC. The structural representation of the conjugation reaction is illustrated below.

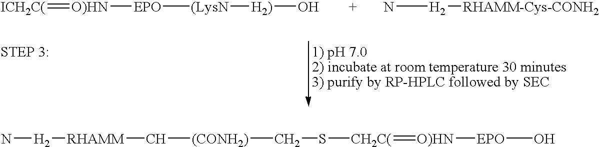

### EXAMPLE 18

Conjugation of RHAMM Peptide and GMCSF by use of a Hetrobifunctional Cross-Linker Sulfo GMBS

GM-CSF stimulates organ and tissue growth, enhancing amino acid uptake and protein synthesis by while reducing protein catabolism. Conjugation of RHAMM to hGH would reduce dosing requirements and toxicity, while increasing target specificity by directing hGH to bone and cartilage.

Preparation of Recombinant GMCSF

The complete coding DNA sequence of human GM-CSF is given as GenBank Accession No. E00951. Recombinant GM-CSF may be purchased from existing commercial sources, or produced by methods known to persons skilled in the art.

The protein is modified through the coupling of a heterobifunctional cross-linker, sulfo-GMBS, to the N-terminal via use of appropriate pH. This cross-linker contains an NHS ester at one end of the molecule and maleimide group at the other end. In preparation of the modified protein, sulfo-GMBS cross-linker is dissolved in 50% acetonitrile and added to GMCSF at pH8 at a molar ratio of 1:3 (i.e., 1 linker:3 protein). Under these pH conditions, the NHS ester group reacts with the N-terminal of the protein. The unreacted maleimide group of the cross-linker is used in later coupling experiments to obtain the conjugate. The preparation of the modified protein is illustrated structurally below.

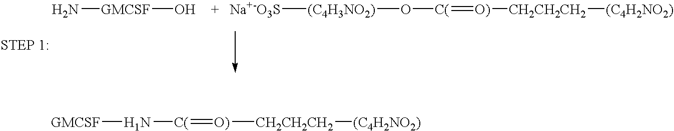

Preparation of RHAMM Peptide

SEQ I.D. 3 is chosen as the RHAMM peptide and is synthesized with a N-terminal protected thiol moiety, which may be accomplished by standard SPPS methods using the following procedure. The RHAMM peptide is synthesized using standard Fmoc SPPS protocols. The last residue to be added is a SAMA group to the RHAMM peptide, followed by cleavage from the resin. Prior to conjugation of the engineered RHAMM peptide with GMCSF, the free thiol on RHAMM must be generated by reaction with hydroxylamine. This is accomplished by dissolving the SAMA-RHAMM peptide in 20 mM phosphate, 150 mM NaCl, 10 mM EDTA at pH 7, followed by addition of hydroxylamine (0.5 M in 20 mM phosphate, 150 mM NaCl, 25 mM EDTA). The preparation of the SAMA-RHAMM peptide and the generation of the free thiol group is illustrated structurally below.

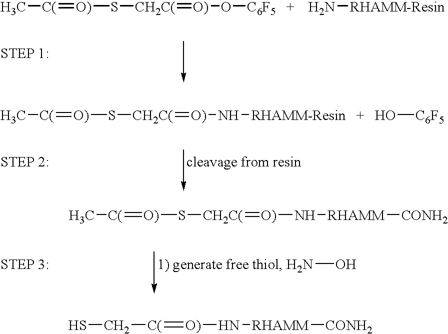

Coupling of RHAMM Peptide and GMCSF

For the coupling of the RHAMM peptide to the N-terminal of the protein, conjugation should take place at a pH of 7 where the coupling occurs through the free thiol group of the N-terminal of the peptide and the unsaturated bond of the maleimide group on the protein (ref). The peptide is dissolved in 20 mM phosphate. 150 mM NaCl, 10 mM EDTA and the protein was buffer exchanged into 20 mM phosphate, 150 mM NaCl, pH7 after the modification reaction. The solution conditions are as follows: incubated at 0° C. for 30 minutes and at room temperature for 45 minutes (ref). The final product is lyophilized and purified by RP-HPLC using water/TFA and acetonitrile/TFA. The product is eluted by buffer exchange into an appropriate buffer using SEC. The coupling reaction between GMCSF and prepared RHAMM peptide is illustrated structurally below.

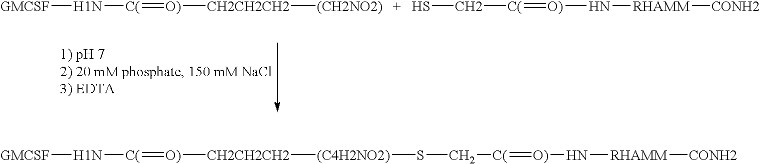

Diagramatic Representation of Reaction Steps

Heterobifunctional Linker Reactions:

1. Modify protein with sulfo-GMBS:

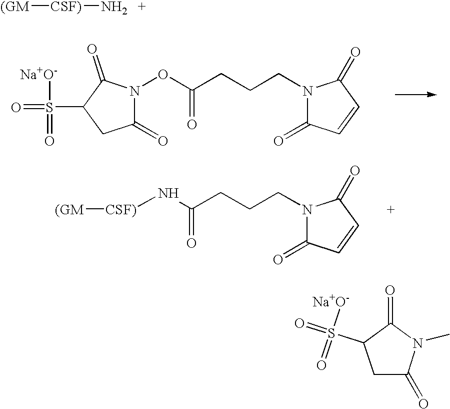

2. Synthesize peptide with SAMA N-terminal:

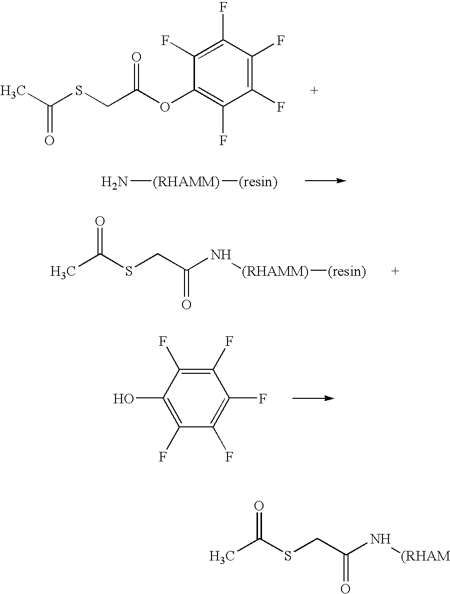

3. Deprotect peptide with hydroxylamine:

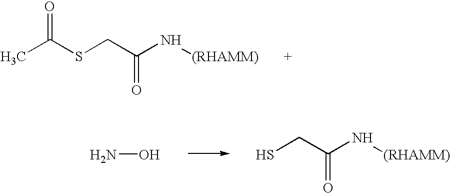

4. Link modified protein to deprotected peptide:

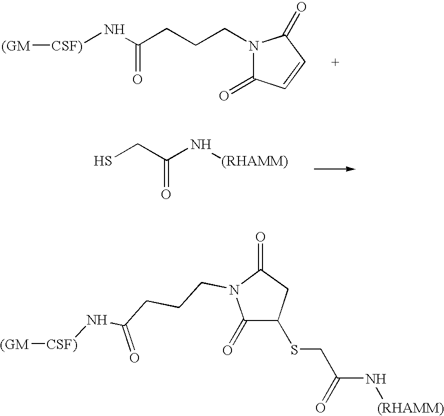

### EXAMPLE 19

Conjugation of RHAMM Peptide and hGH by use of a Hetrobifunctional Cross-Linker Sulfo GMBS

Human growth hormone stimulates organ and tissue growth, enhancing amino acid uptake and protein synthesis by while reducing protein catabolism. Conjugation of RHAMM to hGH would reduce dosing requirements and toxicity, while increasing target specificity by directing hGH to bone and cartilage.

Preparation of Recombinant hGH

The complete coding sequence of human growth hormone is given in. For this method of coupling, the protein does not require modification or engineering prior to the conjugation step. Recombinant hGH may be purchased from existing commercial sources, or produced by methods known to persons skilled in the art.

The protein is modified through the coupling of a heterobifunctional cross-linker, sulfo-GMBS, to the N-terminal via use of appropriate pH. This cross-linker contains an NHS ester at one end of the molecule and maleimide group at the other end. In preparation of the modified protein, sulfo-GMBS cross-linker is dissolved in 50% acetonitrile and added to hGH at pH8 at a molar ratio of 1:3 (i.e., 1 linker:3 protein). Under these pH conditions, the NHS ester group reacts with the N-terminal of the protein. The unreacted maleimide group of the cross-linker is used in later coupling experiments to obtain the conjugate. The preparation of the modified protein is illustrated structurally below.

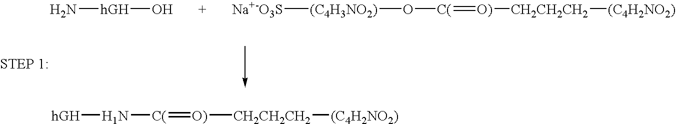

Preparation of RHAMM Peptide

SEQ I.D. 3 is chosen as the RHAMM peptide and is synthesized with a N-terminal protected thiol moiety, which may be accomplished by standard SPPS methods using the following procedure. The RHAMM peptide is synthesized using standard Fmoc SPPS protocols. The last residue to be added is a SAMA group to the RHAMM peptide, followed by cleavage from the resin. Prior to conjugation of the engineered RHAMM peptide with hGH, the free thiol on RHAMM must be generated by reaction with hydroxylamine. This is accomplished by dissolving the SAMA-RHAMM peptide in 20 mM phosphate, 150 mM NaCl, 10 mM EDTA at pH 7, followed by addition of hydroxylamine (0.5 M in 20 mM phosphate, 150 mM NaCl, 25 mM EDTA). The preparation of the SAMA-RHAMM peptide and the generation of the free thiol group is illustrated structurally below.

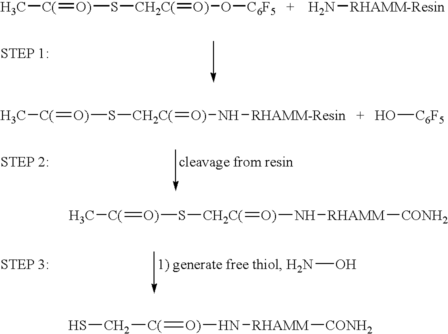

Coupling of RHAMM Peptide and hGH

For the coupling of the RHAMM peptide to the N-terminal of the protein, conjugation should take place at a pH of 7 where the coupling occurs through the free thiol group of the N-terminal of the peptide and the unsaturated bond of the maleimide group on the protein (ref). The peptide is dissolved in 20 mM phosphate, 150 mM NaCl, 10 mM EDTA and the protein was buffer exchanged into 20 mM phosphate, 150 mM NaCl, pH7 after the modification reaction. The solution conditions are as follows: incubated at 0° C. for 30 minutes and at room temperature for 45 minutes (ref). The final product is lyophilized and purified by RP-HPLC using water/TFA and acetonitrile/TFA. The product is eluted by buffer exchange into an appropriate buffer using SEC. The coupling reaction between hGH and prepared RHAMM peptide is illustrated structurally below.

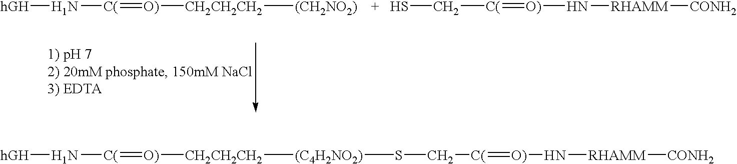

### EXAMPLE 20

Conjugation of PEG-RHAMM Peptide to EPO by use of a Heterobifunctional Cross-Linker Sulfo GMBS

Preparation of Recombinant EPO

The complete coding DNA sequence of human erythropoietin was first disclosed in U.S. patent application Ser. No. 655,841, filed Sep. 28, 1984, which was later issued as U.S. Pat. No. 4,703,008. Recombinant EPO can be produced by methods known to persons skilled in the art, or purchased from existing commercial sources. For this method of coupling, the protein requires modification or engineering prior to the conjugation step.

The protein is modified through the coupling of a heterobifunctional cross-linker, sulfo-GMBS, to the N-terminal via use of appropriate pH. This cross-linker contains an NHS ester at one end of the molecule and maleimide group at the other end. In preparation of the modified protein, sulfo-GMBS cross-linker is dissolved in 50% acetonitrile and added to EPO at pH8 at a molar ratio of 1:3 (i.e., 1 linker:3 protein). Under these pH conditions, the NHS ester group reacts with the N-terminal of the protein. The unreacted maleimide group of the cross-linker is used in later coupling experiments to obtain the conjugate. The preparation of the modified protein is illustrated structurally below.

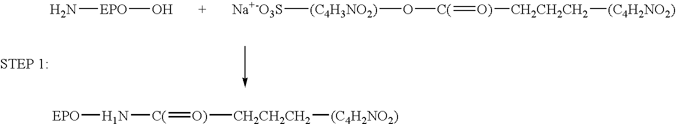

Preparation of RHAMM Peptide

SEQ I.D. 3 is chosen as the RHAMM peptide and is synthesized with a N-terminal protected thiol moiety, which may be accomplished by standard SPPS methods using the following procedure. The RHAMM peptide is synthesized using standard Fmoc SPPS protocols. The last residue to be added is a SAMA group to the RHAMM peptide, followed by cleavage from the resin. Prior to conjugation of the engineered RHAMM peptide with EPO, the free thiol on RHAMM must be generated by reaction with hydroxylamine. This is accomplished by dissolving the SAMA-RHAMM peptide in 20 mM phosphate, 150 mM NaCl, 10 mM EDTA at pH 7, followed by addition of hydroxylamine (0.5 M in 20 mM phosphate, 150 mM NaCl, 25 mM EDTA). The preparation of the SAMA-RHAMM peptide and the generation of the free thiol group is illustrated structurally below.

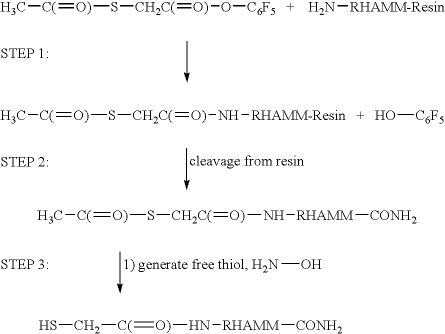

Coupling of RHAMM Peptide and EPO

For the coupling of the RHAMM peptide to the N-terminal of the protein, conjugation should take place at a pH of 7 where the coupling occurs through the free thiol group of the N-terminal of the peptide and the unsaturated bond of the maleimide group on the protein (ref). The peptide is dissolved in 20 mM phosphate, 150 mM NaCl, 10 mM EDTA and the protein was buffer exchanged into 20 mM phosphate, 150 mM NaCl, pH7 after the modification reaction. The solution conditions are as follows: incubated at 0° C. for 30 minutes and at room temperature for 45 minutes (ref). The final product is lyophilized and purified by RP-HPLC using water/TFA and acetonitrile/TFA. The product is eluted by buffer exchange into an appropriate buffer using SEC. The coupling reaction between EPO and prepared RHAMM peptide is illustrated structurally below.

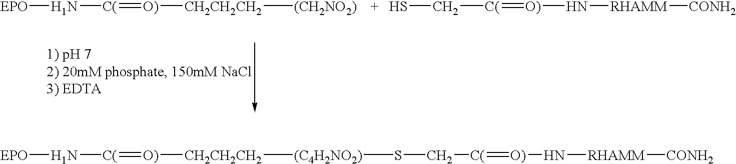

### EXAMPLE 21

Conjugation of RHAMM Peptide to GMCSF by Backbone Engineered Thioether Linkage using a Bifunctional PEG Linkage

GMCSF would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated GM-CSF to a RHAMM peptide will increase the half-life of GM-CSF and direct the conjugate to tissues with a high concentration of HA. It has been reported that approximately 25% of HA is found in bone, which is an optimal target for GM-CSF, and as such, in addition to increasing half-life, conjugation to RHAMM may enhance target specificity.

Preparation of Recombinant GMCSF

The complete coding DNA sequence of human GM-CSF is given as GenBank Accession No. E00951. Recombinant GM-CSF may be purchased from existing commercial sources, or produced by methods known to persons skilled in the art.

A iodoacetyl group must be added to the N-terminal of GMCSF for attachment of the RHAMM peptide. This may be accomplished by adjusting a GMCSF solution (originally in 10 mM phosphate, pH 6.5) to 60 mM phosphate buffer and 0.15% pluronic at a pH of 6.0. lodoacetic anhydride is added to a final 10-fold molar excess, i.e., an equimolar quantity is added in 10 aliqouts at 5 minute intervals at room temperature (ref). Following iodoacetylation, the protein is purified by gel filtration on a PP10 coulumn. The preparation of recombinant GMCSF for thioether linkage conjugation to RHAMM is illustrated structurally below, where H2N— and —H represent the N and C terminals of GMCSF, respectively.

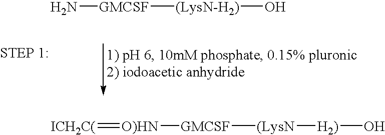

Preparation of C-Terminal PEGylated RHAMM Peptide

SEQ. I.D. 3 comprises the preferred sequence of RHAMM peptide for conjugation to PEG and hGH. The RHAMM peptide must be engineered with a PEG linker molecule and a Cys residue at the C-terminal end of RHAMM, which will later be ligated to the iodoacetylated N-terminal of GMCSF. To this end, a benzhydrylamine resin is utilized to produce a C-terminal amide and the first residue coupled to the resin is the amino acid Cysteine. A bifunctional PEG molecule is then coupled to the Cys residue, followed by RHAMM synthesis as per normal Fmoc SPPS methods (Boc methods are not recommended for this procedure (Lu and Felix, 1993)). Finally, SEC and/or RP-HPLC methods are used to purify the peptide. The process is illustrated structurally below.

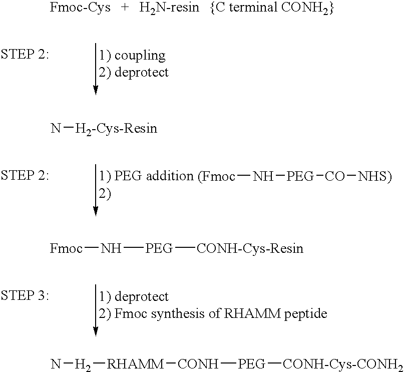

Coupling of RHAMM-PEG to GMCSF

Iodoacetylated GMCSF is buffer exchanged into 10 mM phosphate, pH7. The PEGylated RHAMM-Cys peptide is reconstituted in 10 mM phosphate, pH7 and added to the iodoacetylated GMCSF solution. The solution is allowed to incubate at room temperature for 30 minutes (ref). Purification of the conjugate is by AEX followed by SEC. The structural representation of the conjugation reaction is illustrated below.

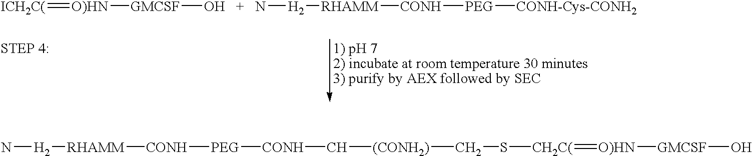

### EXAMPLE 22

Production of RHAMM-GMCSF Direct Conjugate using Backbone Engineered Thioether Linkage: Alternate Method

GMCSF would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated GM-CSF to a RHAMM peptide will increase the half-life of GM-CSF and direct the conjugate to tissues with a high concentration of HA. It has been reported that approximately 25% of HA is found in bone, which is an optimal target for GM-CSF, and as such, in addition to increasing half-life, conjugation to RHAMM may enhance target specificity.

Preparation of Recombinant GMCSF

The complete coding DNA sequence of human GM-CSF is given as GenBank Accession No. E00951. Recombinant GM-CSF may be purchased from existing commercial sources, or produced by methods known to persons skilled in the art.

A iodoacetyl group must be added to the N-terminal of GMCSF for attachment of the RHAMM peptide. This may be accomplished by adjusting a GMCSF solution (originally in 10 mM phosphate, pH 6.5) to 60 mM phosphate buffer and 0.15% pluronic at a pH of 6.0. lodoacetic anhydride is added to a fmal 10-fold molar excess, i.e., an equimolar quantity is added in 10 aliqouts at 5 minute intervals at room temperature (ref). Following iodoacetylation, the protein is purified by gel filtration on a PP10 coulumn. The preparation of recombinant GMCSF for thioether linkage conjugation to RHAMM is illustrated structurally below, where H2N— and —H represent the N and C terminals of GMCSF, respectively.

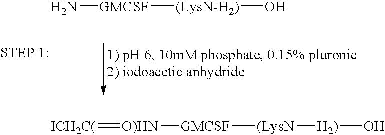

Preparation of RHAMM Peptide

SEQ I.D. 3 is chosen as the RHAMM peptide and is synthesized with a N-terminal protected thiol moiety, which may be accomplished by standard SPPS methods using the following procedure. The RHAMM peptide is synthesized using standard Fmoc SPPS protocols. The last residue to be added is a SAMA group to the RHAMM peptide, followed by cleavage from the resin. Prior to conjugation of the engineered RHAMM peptide with GMCSF, the free thiol on RHAMM must be generated by reaction with hydroxylamine. This is accomplished by dissolving the SAMA-RHAMM peptide in 20 mM phosphate, 150 mM NaCl, 10 mM EDTA at pH 7, followed by addition of hydroxylamine (0.5 M in 20 mM phosphate, 150 mM NaCl, 25 mM EDTA). The preparation of the SAMA-RHAMM peptide and the generation of the free thiol group is illustrated structurally below.

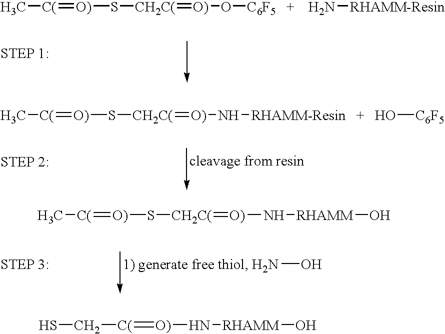

Coupling of the RHAMM Peptide and GMCSF

Iodoacetylated GMCSF is buffer exchanged into 10 mM phosphate, pH 7.0. The SAMA-RHAMM peptide is reconstituted in 10 mM phosphate, pH 7.0 and added to the Iodoacetylated GMCSF solution. The solution is allowed to incubate at room temperature for 30 minutes (reference if any). Purification of the conjugate is by performed by RP-HPLC followed by SEC. The structural representation of the conjugation reaction is illustrated below.

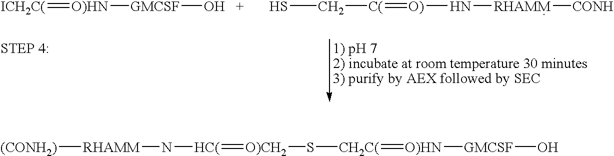

Diagramatic Representation of Reaction Steps

1. Iodoacetylate protein:

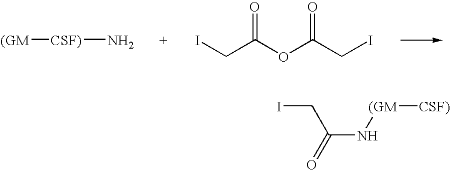

2. Synthesize peptide with SAMA N-terminal:

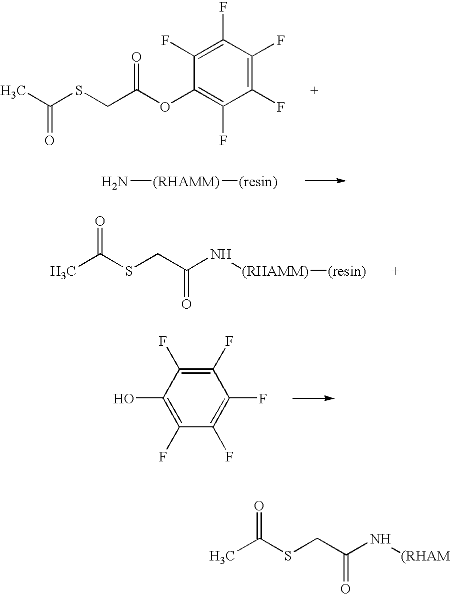

3. Deprotect peptide with hydroxylamine:

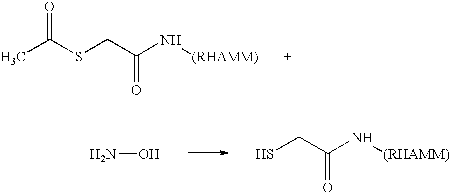

4. Couple modified protein to the free thiol of the peptide:

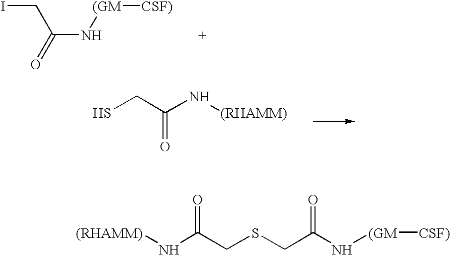

Having illustrated this alternate method for the production of RHAMM-GMCSF direct conjugate using backbone engineered thioether linkage, it should be apparent to a person skilled in the art that this alternate method may be readily modified to conjugate RHAMM to other proteins.

### EXAMPLE 22

Production of RHAMM-GMCSF Direct Conjugate using Backbone Engineered Thioether Linkage: Alternate Method

GMCSF would benefit substantially from the distribution of HA in bone marrow, as targeted by RHAMM peptides. Recombinant erythropoietin is currently produced in the glycosylated form, as the naked form is readily degraded and, therefore, therapeutically ineffective. Therefore, conjugation of non-glycosylated GM-CSF to a RHAMM peptide will increase the half-life of GM-CSF and direct the conjugate to tissues with a high concentration of HA. It has been reported that approximately 25% of HA is found in bone, which is an optimal target for GM-CSF, and as such, in addition to increasing half-life, conjugation to RHAMM may enhance target specificity.

Preparation of Recombinant GMCSF

The complete coding DNA sequence of human GM-CSF is given as GenBank Accession No. E00951. Recombinant GM-CSF may be purchased from existing commercial sources, or produced by methods known to persons skilled in the art.

A iodoacetyl group must be added to the N-terminal of GMCSF for attachment of the RHAMM peptide. This may be accomplished by adjusting a GMCSF solution (originally in 10 mM phosphate, pH 6.5) to 60 mM phosphate buffer and 0.15% pluronic at a pH of 6.0. Iodoacetic anhydride is added to a final 10-fold molar excess, i.e., an equimolar quantity is added in 10 aliqouts at 5 minute intervals at room temperature (ref). Following iodoacetylation, the protein is purified by gel filtration on a PP10 coulumn. The preparation of recombinant GMCSF for thioether linkage conjugation to RHAMM is illustrated structurally below, where

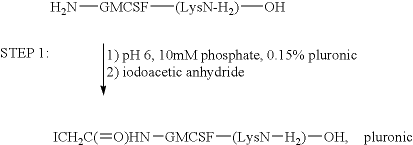

Preparation of RHAMM Peptide

SEQ I.D. 3 is chosen as the RHAMM peptide and is synthesized with a N-terminal protected thiol moiety, which may be accomplished by standard SPPS methods using the following procedure. The RHAMM peptide is synthesized using standard Fmoc SPPS protocols. The last residue to be added is a SAMA group to the RHAMM peptide, followed by cleavage from the resin. Prior to conjugation of the engineered RHAMM peptide with GMCSF, the free thiol on RHAMM must be generated by reaction with hydroxylamine. This is accomplished by dissolving the SAMA-RHAMM peptide in 20 mM phosphate, 150 mM NaCl, 10 mM EDTA at pH 7, followed by addition of hydroxylamine (0.5 M in 20 mM phosphate, 150 mM NaCl, 25 mM EDTA). The preparation of the SAMA-RHAMM peptide and the generation of the free thiol group is illustrated structurally below.

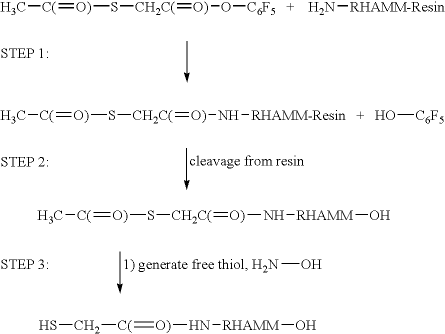

Coupling of the RHAMM Peptide and GMCSF

Iodoacetylated GMCSF is buffer exchanged into 10 mM phosphate, pH 7.0. The SAMA-RHAMM peptide is reconstituted in 10 mM phosphate, pH 7.0 and added to the Iodoacetylated GMCSF solution. The solution is allowed to incubate at room temperature for 30 minutes (reference if any). Purification of the conjugate is by performed by RP-HPLC followed by SEC. The structural representation of the conjugation reaction is illustrated below.

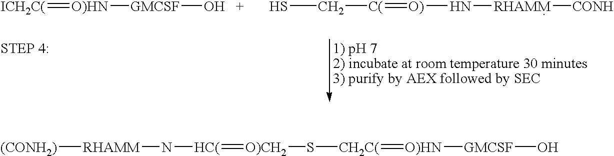

Diagramatic representation of reaction steps

1. Iodoacetylate protein:

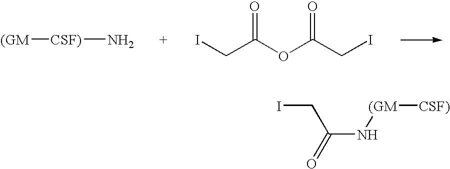

2. Synthesize peptide with SAMA N-terminal:

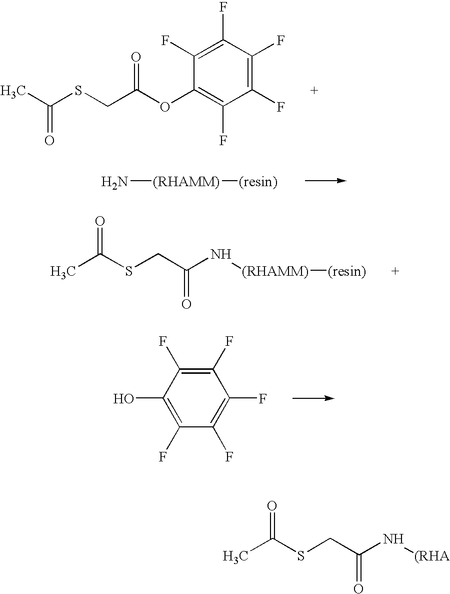

3. Deprotect peptide with hydroxylamine:

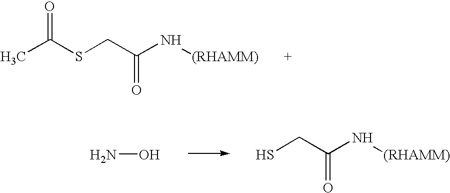

4. Couple modified protein to the free thiol of the peptide:

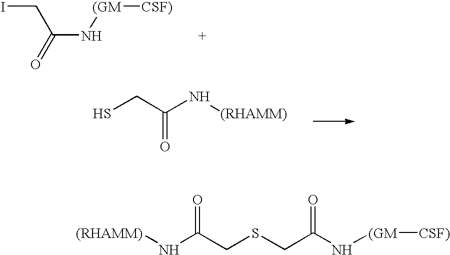

Having illustrated this alternate method for the production of RHAMM-GMCSF direct conjugate using backbone engineered thioether linkage, it should be apparent to a person skilled in the art that this alternate method may be readily modified to conjugate RHAMM to other proteins.

All publications, patents and patent applications referred to herein are incorporated by reference in their entirety to the same extent as if each individual publication, patent or patent application was specifically and individually indicated to be incorporated by reference in its entirety.

Having illustrated and described the principles of the invention in a preferred embodiment, it should be appreciated by those skilled in the art that invention can be modified in arrangement and detail without departure from such principles. We claim all modifications coming within the scope of the following claims.

References

Abrahmsen, L.; Tom, J.; Burnier, J.; Butcher, K. A.; Kossiakoff, A.; and Wells, J. A. Engineering Subtilisin and Its Substrates for Efficient Ligation of Peptide Bonds in Aqueous Solution. Biochemistry. 1991, 30, 4151-4159.

Aoki, F. Y. and Sitar, D. S., Clin Pharm 1988,14:35-51.

Aruffo, A.; Stamenkovic, I.; Melnick, M.; Underhill, C. B.; and Seed, B. CD44 is the Principal Cell Surface Receptor for Hyaluronate. Cell. 1990, 61, 1303-1313.

Asadullah, K.; Docke, W.; Ebeling, M.; Friedrich, M.; Belbe, G.; Audring, H.; Volk, H.; and Sterry, W. Interleukin 10 Treatment of Psoriasis. Archives of Dermatology. 1999, 135(2), 187-192.

Asari, A.; Miyauchi, S; Kuriyama, S; Machida, A; Kohno, K; Uchiyama, Y. Localization of Hyaluronic Acid in Human Articular Cartilage. Journal of Histochemistry and Cytochemistry. 1994, 42(4), 513-522.

Baca, M.; Muir, T. W.; Schnolzer, M.; and Kent, B. H. Chemical Ligation of Cysteine-Containing Peptides: Synthesis of a 22 kDa Tethered Dimer of HIV-1 Protease. Journal of the American Chemical Society. 1995, 117, 1881-1887.

Blake, J. Peptide Segment Coupling in Aqueous Medium: Silver Ion Activation of the Thiolcarboxyl Group. International Journal of Peptide and Protein Research. 1981, 17, 273-274.

Blake, J.; Total Synthesis of S-carbamoylmethyl Bovine Apocytochrome c by Segment Coupling. International Journal of Peptide and Protein Research. 1986, 191-200.

Blake, J. and Li, C. H. New Segment-coupling Method for Peptide Synthesis in Aqueous Solution: Apllication to Synthesis of Human [Gly17]-_-endorphin. Proceedings of the National Academy of Science. 1981, 78(7), 4055-4058.

We clam:

1. A conjugate comprising an HA-binding protein contiguous with, or coupled to a polypeptide conjugated to a therapeutic agent.

2. A conjugate comprising an HA-binding peptide conjugated to a therapeutic agent.

3. An isolated and purified nucleic acid sequence encoding an HA-binding protein or peptide in sequence with a therapeutic agent.

4. An isolated and purified nucleic acid sequence according to claim 3 wherein the nucleic acid sequence does not encode an intervening amino acid sequence between the HA-binding peptide in sequence and the therapeutic agent.

5. A conjugate according to any one of claims 1-4 wherein the HA-binding protein is a receptor for hyaluronan-mediated motility (RHAMM), or a variant thereof capable of binding HA, or an anti-HA antibody or variant thereof capable of binding HA.

6. A conjugate according to claim 5 wherein the HA-binding protein is a polypeptide comprising an amino acid sequence of SEQ ID NO. 1, SEQ ID NO. 2 or SEQ ID NO. 3, or a fragment, analog or derivative thereof which maintains the ability to bind HA.

7. A conjugate according to claim 5 wherein the HA-binding protein has an amino acid sequence comprising a structure of B¹-Aₙ-B² wherein B¹ and B² are the same or different basic amino acid residues and Aₙ is an amino acid sequence containing seven or eight amino acid resides which are the same or different and are neutral or basic amino acids.

8. A conjugate according to claim 7 wherein the HA-binding peptide comprises an amino acid sequence selected from the group consisting of KQKKHXXK, KIHXXKUK, KLRsQLXKR and KLRSQLXKRK wherein
each X is independently selected from V or D

9. A conjugate according to claim 8 wherein the HA-binding peptide comprises an amino acid sequence according to one of SEQ. ID. NO.: 7, SEQ. ID. NO.: 8, SEQ. ID. NO.: 9, or SEQ. ID. NO. 10.

10. A conjugate according to claim 5 wherein the HA binding peptide has an amino acid sequence comprising the following Formula I:
X₁ —X₂—X₁ —X₃—X₄—X₃—X₄—X₃—X₃—X₃—X₅—X₆—X₆—X₆—X₁
wherein:
each X₁ is independently selected from a hydroxy amino acid residue;
each X₂ is independently selected from a sulfur containing amino acid residue;
each X₃ is independently selected from a basic amino acid residue;
each X₄ is independently selected from an imino or aromatic amino acid residue;
each X₅ is independently selected from a dicarboxylic acid amino acid residue; and
each X₆ is independently selected from an aliphatic amino acid residue; and preferably:
each X₁ is independently selected from threonine or serine;
each X₂ is independently selected from methionine or cysteine;
each X₃ is independently selected from arginine, lysine or histidine;
each X₄ is independently selected from proline, phenylalanine or tryptophan;
each X₅ is independently selected from asparagine or glutamine; and
each X₆ is independently selected from leucine, isoleucine, valine or alanine.

11. A conjugate according to claim 10 wherein the amino acid sequence of Formula I is TMTRPHFHKRQLVLS (SEQ. ID. NO.: 11).

12. A conjugate according to claim 5 wherein the HA binding peptide has an amino acid sequence comprising the following Formula II:
Y₁—Y₁—Y₂—Y₂—Y₁—Y₃—Y₁—Y₃—Y₃—Y₁—Y₃—Y₁—Y₂—Y₃—Y₃
wherein:
each Y₁ is independently selected from a hydroxy amino acid residue;
each Y₂ is independently selected from a sulfur containing amino acid residue; and
each Y₃ is independently selected from a basic amino acid residue; and preferably:
each Y₁ is independently selected from serine or threonine;
each Y₂ is independently selected from methionine or cysteine; and
each Y₃ is independently selected from arginine, lysine or histidine.

13. A conjugate according to claim 12 wherein the amino acid sequence of the Formula II is STMMSRSHKTRSCHH (SEQ. ID. NO.: 12).

14. A conjugate according to claim 5 wherein the HA binding peptide has an amino acid sequence comprising the following Formula III:
Z₁-Z₁-Z₂-Z₂-Z₁-Z₃-Z₁-Z₃-Z₃-Z₁-Z₃-Z₁-Z₃-Z₃
wherein:
each Z₁ is independently selected from a hydroxy amino acid residue;
each Z₂ is independently selected from a sulfur containing amino acid residue; and
each Z₃ is independently selected from a basic amino acid residue, and fragments; and preferably,
each Z₁ is independently selected from serine or threonine;
each Z₂ is independently selected from methionine or cysteine; and
each Z₃ is independently selected from arginine, lysine or histidine.

15. A conjugate according to claim 14 wherein the amino acid sequence of the Formula III is STMMSRSHKTRSHH (SEQ. ID. NO.: 13).

16. A conjugate according to claim 15 wherein a valine residue is placed at the C-terminal and obtains the following sequence: STMMRSHKTRSHHV (SEQ. ID. NO.: 14).

17. A conjugate according to any of claims 1-16 wherein the therapeutic agent is selected from the group comprising a colony stimulating factor (G-CSF, GM-CSF or M-CSF), erythropoietin, growth hormone, parathyroid hormone, an interferon, an interleukin, an antibody, an immunoadhesin, or any functional fragment of any of the forgoing.

18. A method of preparing a conjugate according to any one of claims 5-17 comprising the following steps: (i) inserting a first nucleotide sequence encoding a HA binding protein directly linked to a second nucleotide sequence encoding a therapeutic protein into a suitable vector; (ii) expressing the vector in an acceptable host; and (iii) purifying said conjugate molecule from said host or expression medium.

19. A method according to claim 18 wherein the first nucleotide sequence encodes an HA binding peptide.

20. A method according to claim 18 or 19 wherin the first nucleotide sequence is indirectly linked to the second nucleotide sequence.

21. A process for preparing a pharmaceutical for treating an animal in need of treatment comprising the steps of preparing a purified conjugate according to anyone of claims 1-17 and suspending the conjugate molecule in a pharmaceutically acceptable carrier, diluent or excipient.

22. A pharmaceutical composition comprising purified conjugate according to anyone of claims 1-17 suspended in a pharmaceutically acceptable carrier, diluent, or excipient.

23. A method for altering in vivo the distribution of a therapeutic agent comprising conjugating the therapeutic agent to form a conjugate according to any one of claims 1-17, administering the conjugate to the animal where the conjugate molecule will distribute primarily in tissues and organs containing high levels of endogenous HA.

24. A method of treating a mammal with a disorder where a diseased tissue/organ of the mammal contains high levels of HA comprising administering a conjugate according to anyone of claims 1-17 to said mammal.
